SEQUENCE LISTING (85 sequences)

SEQ ID NO: 1 - DNA, 4680 bp, Homo sapiens
  source 1..4680: mol_type = genomic DNA
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacaggtaggatcccctccgtgccctgtgatgccttctctccctcactttgagcatgtggttggggcgcgggtggtgggccctggggtgaattatgaaggcggtggcagtcctcgcatgcctgagtgccctgttctgcactcctctcctccccaccctgcccatattcttcacaccctccttccctccccagtatcaaaacctaccctcagactctttctgcatgtggacatgtaaggggctggtggactctggttgcattggaagggaaggagtgctaacagtggcatcccaggcactagctggcagttgggggaagctttggggggcactggcactggtaatagcctctgaaattataagccactaattatgagcccctacagttataaaggaggaaagaacctgaggatgttcatctgcatctttggggcactctctcccctgctctgaaggtccccttgtcctcagctcttgatgggaggtgaggagcgtagagatttccgttgctgagttgaggggaattgacacaccaaatttttgtacacaattgcttcttcccgctgaggaaagcgcaccttttgctcccccagagcactggacaaacctgggcaagaggagagacggtgccaaatggagtcttgttcctgcagcttcttagatggtggtcagggggaaggcggggttctgaggcatggatgggaggtggtcagatgggagagggccacggcccatctggtctctctagttccaaaaggcaagccaccaacttcccaatcctatctttcaagcctctgtcacagtaatggttgaagatggcaggcaagggaggaccaagagagaagtcaaagcagggggctctgggggctgccccagcaacaggctggtgctctaagcccatctcccccgccctaaggagggttcccaaaatagcagcctcatgtctcccccaaaatatctccgagacgggtccttcctgaaaggggaacaaagccacagaaatagggaagctggaagttaaaggtcaggaaagtcggtaccaatgtgggcggctgcagagcaagcaagagtggcggggcagggagagccagccccaggccgagaggagaggtcccagtcccaaatgatggcaaagagatgtgcagaacagaactggagggggttttaagaccaagtgcctccagaatagacccagagaaggtcagttacttctccaaggaggcccagcaagagcaacagtgagaaaaccaaacccaggtcccaggtttcctggttcccaatttcccacaaacacatgctgtgccatccgctcccaacttgtataagaacctaagctccttcagggcaggatttttgtctgtttaggtcactgcacagagcaagtgttcaaagcgtatttggtaaacaaaggagtgaatgaatgtctcctccccagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcaggtgagtgggcagtggctgcctctctggcctgcccctgttccaggcctcatgttttgggcgtctgtgtgcagaaccacccactgggctctcagggatcctgggaaaacccatcgtatggtctcatctccctactcagggctaccaaggatagttgtgcaggttgctcactatacaagggggccaaaatccagcccggactccttttgccagactgtgttccctggtgcagagctgcatcacctgcaagaagggcacctttctctactttgcattaaggcactctgtgaactagcctggccttggccctccttgcccaaacagctttatccatctccatccagttagcatgtgaacatgataggaggtccttaaggaggaggagattaatttattccctcaccaaccaaccaatggccctatcaacgtaagtgaagcaccaacctggtgcagaaaatcacccctagaaaggaacactgcttgtgctgggcaatgtgcttcacttctctgggcctccattctctcatctgtaaatggagagggtggtgtctgtcctgctgaccccatagggttcctgtgagtttttatttggcggggggcgttgggggcagggtgtgacatgtgctccaagcctcctgctactgaactgtcttgggatcgtcattccaggtgtgagtaggtgggccagggtgtctcctttccacccccgagctctgcgcagggaccaccctccaggaaggccttctgcttgagtctgcagaacacaactgtgtccccaaaggagaacaaatctggggctacaaaggccttttctgactctcctcctttccaaccccttctcccagtatggctctttaccatggaaatgaggtcaggagtagtatttgcctgacttggggaggccaggggagactgccagtcatagacacaaaccccacctccaccactttcctaaggatggccggtggcagtgaactcccttagccacctcccaagagggagacagaaaaataaaagtagcccatagcttgtgtagacagcttgacagtttagataggattttcaagtacatccacgattgtatttgctcttcttagcccctgtgcggaaggaggggggcattgcagttactgagctccgatgaggtgctggatacggcacagaggtgccctcacggaatcctggcaaccctgctattaaggaagtgttcctaacggcccactttataagtggggaaactgaggcttatggagtgtaatgacctgatccacataggaccagtgcccaggggacctcaccccacccataaaggttctcccgccaaggttctccccctgcctcgaggaggaaggggctggaggctcatggctctgcctcccatagaccccctggatcaccgggatgctggagatctctggttccccggggagtctgagagcttcgaggatgcccatgtggagcacagcatctccaggtacagcgatgggtctttcctaaaacccgtgggcagacttccaccctgcagagccccacgggggtgggggctgggcatcctgaactacaaattacaaggcaactagagcctgaagggccctggatatctcttatgtaaacacccctccaccagctgctacatgttagttagttagcgtgactgtgtcagttgttcatttattggaagacttccataatgctttgtaaagagcagttgctctacgactcctgaatatcagctcccgcccctcccctggcagcccccagacatccccataattcagcaacaatgaatgttttagtactttagtacaaagctaagacccccagatctctgcttcattggtaggaccagaatcagttctgactggtccatgcctgtgtactggtgtttaaacgttgttcatataaccacatgtgtgtacacacacacacccatggcatagcactgtatacagaagaaaactgctcagtgagggaaggtggtgtgtttaagtgcactcagcaggttagtggcagagcatgcctagaactcaagtctcttgactatgggctcccaaagctccaaaaaggcccatcccagggatggtgctgggtgggatttgctgaggtcctagaagctcctggtttctccccattccctgagagaagattctaatctgtaggccatctcaaggctcccatctgccttcttgtctctccaaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccaggtgggcttggagagcacagccatcaagggtggactgggcagggatgggaggcggtgccgagaggggccacatgagtccttggactattgcagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggat

SEQ ID NO: 2 - DNA, 4680 bp, Homo sapiens
  source 1..4680: mol_type = genomic DNA
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacaggtaggatcccctccgtgccctgtgatgccttctctccctcactttgagcatgtggttggggcgcgggtggtgggccctggggtgaattatgaaggcggtggcagtcctcgcatgcctgagtgccctgttctgcactcctctcctccccaccctgcccatattcttcacaccctccttccctccccagtatcaaaacctaccctcagactctttctgcatgtggacatgtaaggggctggtggactctggttgcattggaagggaaggagtgctaacagtggcatcccaggcactagctggcagttgggggaagctttggggggcactggcactggtaatagcctctgaaattataagccactaattatgagcccctacagttataaaggaggaaagaacctgaggatgttcatctgcatctttggggcactctctcccctgctctgaaggtccccttgtcctcagctcttgatgggaggtgaggagcgtagagatttccgttgctgagttgaggggaattgacacaccaaatttttgtacacaattgcttcttcccgctgaggaaagcgcaccttttgctcccccagagcactggacaaacctgggcaagaggagagacggtgccaaatggagtcttgttcctgcagcttcttagatggtggtcagggggaaggcggggttctgaggcatggatgggaggtggtcagatgggagagggccacggcccatctggtctctctagttccaaaaggcaagccaccaacttcccaatcctatctttcaagcctctgtcacagtaatggttgaagatggcaggcaagggaggaccaagagagaagtcaaagcagggggctctgggggctgccccagcaacaggctggtgctctaagcccatctcccccgccctaaggagggttcccaaaatagcagcctcatgtctcccccaaaatatctccgagacgggtccttcctgaaaggggaacaaagccacagaaatagggaagctggaagttaaaggtcaggaaagtcggtaccaatgtgggcggctgcagagcaagcaagagtggcggggcagggagagccagccccaggccgagaggagaggtcccagtcccaaatgatggcaaagagatgtgcagaacagaactggagggggttttaagaccaagtgcctccagaatagacccagagaaggtcagttacttctccaaggaggcccagcaagagcaacagtgagaaaaccaaacccaggtcccaggtttcctggttcccaatttcccacaaacacatgctgtgccatccgctcccaacttgtataagaacctaagctccttcagggcaggatttttgtctgtttaggtcactgcacagagcaagtgttcaaagcgtatttggtaaacaaaggagtgaatgaatgtctcctccccagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcaggtgagtgggcagtggctgcctctctggcctgcccctgttccaggcctcatgttttgggcgtctgtgtgcagaaccacccactgggctctcagggatcctgggaaaacccatcgtatggtctcatctccctactcagggctaccaaggatagttgtgcaggttgctcactatacaagggggccaaaatccagcccggactccttttgccagactgtgttccctggtgcagagctgcatcacctgcaagaagggcacctttctctactttgcattaaggcactctgtgaactagcctggccttggccctccttgcccaaacagctttatccatctccatccagttagcatgtgaacatgataggaggtccttaaggaggaggagattaatttattccctcaccaaccaaccaatggccctatcaacgtaagtgaagcaccaacctggtgcagaaaatcacccctagaaaggaacactgcttgtgctgggcaatgtgcttcacttctctgggcctccattctctcatctgtaaatggagagggtggtgtctgtcctgctgaccccatagggttcctgtgagtttttatttggcggggggcgttgggggcagggtgtgacatgtgctccaagcctcctgctactgaactgtcttgggatcgtcattccaggtgtgagtaggtgggccagggtgtctcctttccacccccgagctctgcgcagggaccaccctccaggaaggccttctgcttgagtctgcagaacacaactgtgtccccaaaggagaacaaatctggggctacaaaggccttttctgactctcctcctttccaaccccttctcccagtatggctctttaccatggaaatgaggtcaggagtagtatttgcctgacttggggaggccaggggagactgccagtcatagacacaaaccccacctccaccactttcctaaggatggccggtggcagtgaactcccttagccacctcccaagagggagacagaaaaataaaagtagcccatagcttgtgtagacagcttgacagtttagataggattttcaagtacatccacgattgtatttgctcttcttagcccctgtgcggaaggaggggggcattgcagttactgagctccgatgaggtgctggatacggcacagaggtgccctcacggaatcctggcaaccctgctattaaggaagtgttcctaacggcccactttataagtggggaaactgaggcttatggagtgtaatgacctgatccacataggaccagtgcccaggggacctcaccccacccataaaggttctcccgccaaggttctccccctgcctcgaggaggaaggggctggaggctcatggctctgcctcccatagaccccctggatcaccgggatgctggagatctctggttccccggggagtctgagagcttcgaggatgcccatgtggagcacagcatctccaggtacagcgatgggtctttcctaaaacccgtgggcagacttccaccctgcagagccccacgggggtgggggctgggcatcctgaactacaaattacaaggcaactagagcctgaagggccctggatatctcttatgtaaacacccctccaccagctgctacatgttagttagttagcgtgactgtgtcagttgttcatttattggaagacttccataatgctttgtaaagagcagttgctctacgactcctgaatatcagctcccgcccctcccctggcagcccccagacatccccataattcagcaacaatgaatgttttagtactttagtacaaagctaagacccccagatctctgcttcattggtaggaccagaatcagttctgactggtccatgcctgtgtactggtgtttaaacgttgttcatataaccacatgtgtgtacacacacacacccatggcatagcactgtatacagaagaaaactgctcagtgagggaaggtggtgtgtttaagtgcactcagcaggttagtggcagagcatgcctagaactcaagtctcttgactatgggctcccaaagctccaaaaaggcccatcccagggatggtgctgggtgggatttgctgaggtcctagaagctcctggtttctccccattccctgagagaagattctaatctgtaggccatctcaaggctcccatctgccttcttgtctctccaaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccaggtgggcttggagagcacagccatcaagggtggactgggcagggatgggaggcggtgccgagaggggccacatgagtccttggactattgcagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggat

SEQ ID NO: 3 - RNA, 860 nt, Homo sapiens
  source 1..860: mol_type = mRNA
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggat

SEQ ID NO: 4 - RNA, 1012 nt, Homo sapiens
  source 1..1012: mol_type = mRNA
ccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggccatctcaaggctcccatctgccttcttgtctctccaaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggata

SEQ ID NO: 5 - RNA, 983 nt, Homo sapiens
  source 1..983: mol_type = mRNA
actctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctgga

SEQ ID NO: 6 - RNA, 789 nt, Homo sapiens
  source 1..789: mol_type = mRNA
actctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctgga

SEQ ID NO: 7 - RNA, 1098 nt, Homo sapiens
  source 1..1098: mol_type = mRNA
ccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggccatctcaaggctcccatctgccttcttgtctctccaaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggataaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - RNA, 867 nt, Homo sapiens
  source 1..867: mol_type = mRNA
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggaaaaaaaaa

SEQ ID NO: 9 - RNA, 1041 nt, Homo sapiens
  source 1..1041: mol_type = mRNA
tgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttaaaaa

SEQ ID NO: 10 - RNA, 770 nt, Homo sapiens
  source 1..770: mol_type = mRNA
tcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtga

SEQ ID NO: 11 - RNA, 1044 nt, Homo sapiens
  source 1..1044: mol_type = mRNA
tgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggat

SEQ ID NO: 12 - RNA, 860 nt, Homo sapiens
  source 1..860: mol_type = mRNA
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggat

SEQ ID NO: 13 - RNA, 1012 nt, Homo sapiens
  source 1..1012: mol_type = mRNA
ccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggccatctcaaggctcccatctgccttcttgtctctccaaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggata

SEQ ID NO: 14 - RNA, 983 nt, Homo sapiens
  source 1..983: mol_type = mRNA
actctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctgga

SEQ ID NO: 15 - RNA, 789 nt, Homo sapiens
  source 1..789: mol_type = mRNA
actctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctgga

SEQ ID NO: 16 - RNA, 1098 nt, Homo sapiens
  source 1..1098: mol_type = mRNA
ccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggccatctcaaggctcccatctgccttcttgtctctccaaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggataaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - RNA, 867 nt, Homo sapiens
  source 1..867: mol_type = mRNA
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggaaaaaaaaa

SEQ ID NO: 18 - RNA, 1041 nt, Homo sapiens
  source 1..1041: mol_type = mRNA
tgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttaaaaa

SEQ ID NO: 19 - RNA, 770 nt, Homo sapiens
  source 1..770: mol_type = mRNA
tcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtga

SEQ ID NO: 20 - RNA, 1044 nt, Homo sapiens
  source 1..1044: mol_type = mRNA
tgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggat

SEQ ID NO: 21 - DNA, 860 bp, Homo sapiens
  source 1..860: mol_type = other DNA
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggat

SEQ ID NO: 22 - DNA, 1012 bp, Homo sapiens
  source 1..1012: mol_type = other DNA
ccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggccatctcaaggctcccatctgccttcttgtctctccaaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggata

SEQ ID NO: 23 - DNA, 983 bp, Homo sapiens
  source 1..983: mol_type = other DNA
actctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctgga

SEQ ID NO: 24 - DNA, 789 bp, Homo sapiens
  source 1..789: mol_type = other DNA
actctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctgga

SEQ ID NO: 25 - DNA, 1098 bp, Homo sapiens
  source 1..1098: mol_type = other DNA
ccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggccatctcaaggctcccatctgccttcttgtctctccaaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggataaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - DNA, 867 bp, Homo sapiens
  source 1..867: mol_type = other DNA
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggaaaaaaaaa

SEQ ID NO: 27 - DNA, 1041 bp, Homo sapiens
  source 1..1041: mol_type = other DNA
tgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttaaaaa

SEQ ID NO: 28 - DNA, 770 bp, Homo sapiens
  source 1..770: mol_type = other DNA
tcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtga

SEQ ID NO: 29 - DNA, 1044 bp, Homo sapiens
  source 1..1044: mol_type = other DNA
tgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggat

SEQ ID NO: 30 - DNA, 860 bp, Homo sapiens
  source 1..860: mol_type = other DNA
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggat

SEQ ID NO: 31 - DNA, 1012 bp, Homo sapiens
  source 1..1012: mol_type = other DNA
ccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggccatctcaaggctcccatctgccttcttgtctctccaaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggata

SEQ ID NO: 32 - DNA, 983 bp, Homo sapiens
  source 1..983: mol_type = other DNA
actctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctgga

SEQ ID NO: 33 - DNA, 789 bp, Homo sapiens
  source 1..789: mol_type = other DNA
actctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctgga

SEQ ID NO: 34 - DNA, 1098 bp, Homo sapiens
  source 1..1098: mol_type = other DNA
ccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggccatctcaaggctcccatctgccttcttgtctctccaaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggataaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - DNA, 867 bp, Homo sapiens
  source 1..867: mol_type = other DNA
gggcagcgcctgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggaaaaaaaaa

SEQ ID NO: 36 - DNA, 1041 bp, Homo sapiens
  source 1..1041: mol_type = other DNA
tgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttaaaaa

SEQ ID NO: 37 - DNA, 770 bp, Homo sapiens
  source 1..770: mol_type = other DNA
tcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtga

SEQ ID NO: 38 - DNA, 1044 bp, Homo sapiens
  source 1..1044: mol_type = other DNA
tgacatgcctgatcctctcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgtaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctggat

SEQ ID NO: 39 - protein, 219 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRAERHVKEDDGRKSPGEVPPGTSPACILATWPPGLLVLLWQETTLPEHCFSWTLEAGTG

SEQ ID NO: 40 - protein, 222 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRPSQGSHLPSCLSKEPLGRRNPLPTHFHPSPPGLHLSHQDSSSQRPLGCSLAWTEARDTSTQ

SEQ ID NO: 41 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 42 - protein, 249 aa, Homo sapiens
MPDPLFSAVQGKDEILHKALCFCPWLGKGGMEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRAERHVKEDDGRKSPGEVPPGTSPACILATWPPGLLVLLWQETTLPEHCFSWTLEAGTG

SEQ ID NO: 43 - protein, 32 aa, Homo sapiens
MEPLRLLILLFVTELSGAHNTTVFQGVAGQSL

SEQ ID NO: 44 - protein, 62 aa, Homo sapiens
MPDPLFSAVQGKDEILHKALCFCPWLGKGGMEPLRLLILLFVTELSGAHNTTVFQGVAGQSL

SEQ ID NO: 45 - DNA, 3895 bp, Homo sapiens
  source 1..3895: mol_type = other DNA
acttgcctggacgctgcgccacatcccaccggcccttacactgtggtgtccagcagcatccggcttcatggggggacttgaaccctgcagcaggctcctgctcctgcctctcctgctggctgtaagtggtgagttaggggcttccgtggctgcctcccgggtccctgggctcagcttggggcagggcagggagtggggtggaacgagagaaccaaaagtgggtgttgggatgggagcaggtccccaacctcccaaagcctgtgggtttctcccagagcccaagcccccaagttttgtcgtccgctacaagcaggggagaagagacatctaagtgtgttgccacaggacaagttgtgcagaagtaacgcacatagtccggtggcccagacgccagccccctgagtcccgccagacacgctctcccccttgctaacctcttggctgtcaggatccaccttccctggcttctaaacttgcctcccccacccccgtcataactctgtgcctcagtttaccttctttttcctcctcaggtctccgtcctgtccaggcccaggcccagagcggtaggcctagacccagcagtccctctctctacctcccagagacctccctgtctccgtctctcccacaccctttccaaacctccctgccgctgacccccctccccacagttcccagcacacactgacctcccctgacccctgtgctgcagattgcagttgctctacggtgagcccgggcgtgctggcagggatcgtgatgggagacctggtgctgacagtgctcattgccctggccgtgtacttcctgggccggctggtccctcgggggcgaggggctgcggagggtgagtggggctagcaggggacatcctgaggacttgcctagatgggggtggggggctgggtaaactcccagatctcaaacatccaaagggatggtaatggaggtgctgatttggaatgacaaaacaccctcaactcaccactctgcctgtctctgcaccccacaacacccctcacctccagcagcgacccggaaacagcgtatcactgagaccgagtcgccttatcaggtgagaaaaccagactgtcctcctcacatttcactcctgccagggatatctgccccagatactccttcatgccatcagagggacctcaaagccactctgcctaaagcacccccaaaccttgaaggcacatgggacatctgtccaaaactgggccgaatgatcaaccacaagcttatggtcagcttaaaatgtgtgaagaggggctgggcacggtgactcaaacctgtaatcccagcactttgggagacagaggcgggtggatcacttgaggtcaggagttcgagaccaacatggtgaaaccctgtctctactaaaaatacaaaattagctgggcgtggtaacgcatgcctgtaatcccagctacttgggaggctgaggcaggagaatcctttgacccaggaggcggaggttgcagtgagccaagatcacgcaattgcactccagcctgggcaacaagaggaaaactctgtcaaaaaaaaaaaagcgtgaagagttaaaaagaaaaaaaaaagggtggctttgcctttgtaagtgtaaacacaggccttgccagaagtcaggccaggaattttctggaagaaacccagccctaggacgactgatttctgggagacgctctcagtagggcagagaatgtcattcagccctaccttggtaactctaaagagaggagtcctagaatctcttttttgggtttgaggacccttcatccctttgagaaactaagaaaatctagactcccagaaaaatctacacaaaaaatacaaaattgtgcatataatctgagagcagtgtaaatgttctttataattaaaaaaaatttttttttggccaggcatggtggcacacacctgtaatcccagcactttgggaggccaaggtgggtggatcacgaggtcaggagttcaagaccagccaggccaggatggtgaaaccccatctctactaaaaatacaaaaattagctgggcatggtgtcatgcgcctgtaattccagcttctcaggaggctgaggcaagagaatcgcttgaacccagggggcggaggttgcagtgagcctgggcgacagagtgagactccatctcaaaaaaaaaaaaaagacaggggtctcactatgttgccccagctggtctcaaactcccagtctcaagtgatcctcccgcctcagcctccgaaaatcttgggattacaggctcaagcaaccatgctggttgagtattcttgaagctcttttttctttctctactttaatttttttttttttttgagatggagtctcattctgtcacccaggctggagtgcaatggtgtggtctcagctcactgcaacctccacctcccaggttcaagcgattctcttgcctgagcctcccgagtagctggaactacaggtgcatgccaccacacccggctaatttttgtatttttagtagagatggggtttcactatgttggccaggctggtctcaatctcctgacctcgtgatccgcccacctttccctcccaaagtgctgggattagaggtgtgagccaccgcgcccggccttaattttttttttttttttaaagtagagataggttctcactatgttgcccaggctggtctcaaactcctgggctcaagagatcctgttacctcggtctcccaaagtgctgggattataggcatgagtcaccatgcctggctgaagctctttttaaaaactatagagtatacaagagaatctagtcatctatccccctctctgtcaatgttcctttttaaccctttagggggtcatgagattctctaagacctctcccctgaagaatgcacgcccagactagtcatcatatatgattttagggtttctgtctgaggcctgcccctgggggccacatagatcctccccctaccccaagtgtgaacactgctgtgtgaagtctgatatattgtctgcaaccagctttgcctattcacctcacttgtggataaagtttctttgtgaagtctaagaatctcaggtttggctctgtgatatccagaagaccgaggggcccttccagttactggtggcagtgaaattttagggcaaagcattacccggatgtatttattccctctgtggatgtgtctagcacctataagtgctaaatgctggggacacagtgacagagtttatatcctagttaagacaaacttaaagcaaatgaacaaataaaataattttgggaggcacagtaaaggaaataaacaggcttttttttttttttttacagtggctggagtgtggggggaagaaactacagaggggaaattgagaaatgcctctctgaggaggtgacgtgagctgagacttgaagggagaggggatctaggggaacagagttttaggcagaggaaacagcacacgcagggaccctgaggctggggcacactgatgaggcagaggagaagggggaacattgaatgtacttgaataaaatccggatctctttcatggctataaggctgaagaagctgccaggggccttgcaggccactgggagaaggctggattttatttggtgggtcccttccactgatggcccccattcctttaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaaca

SEQ ID NO: 46 - RNA, 541 nt, Homo sapiens
  source 1..541: mol_type = mRNA
acttgcctggacgctgcgccacatcccaccggcccttacactgtggtgtccagcagcatccggcttcatggggggacttgaaccctgcagcaggctcctgctcctgcctctcctgctggctgtaagtgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaaca

SEQ ID NO: 47 - RNA, 415 nt, Homo sapiens
  source 1..415: mol_type = mRNA
acttgcctggacgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatc

SEQ ID NO: 48 - RNA, 620 nt, Homo sapiens
  source 1..620: mol_type = mRNA
tccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggggatggtaatggaggtgctgatttggaatgacaaaacaccctcaactcaccactctgcctgtctctgcaccccacaacacccctcacctccagcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaa

SEQ ID NO: 49 - RNA, 522 nt, Homo sapiens
  source 1..522: mol_type = mRNA
acatcccaccggcccttacactgtggtgtccagcagcatccggcttcatggggggacttgaaccctgcagcaggctcctgctcctgcctctcctgctggctgtaagtgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaa

SEQ ID NO: 50 - RNA, 575 nt, Homo sapiens
  source 1..575: mol_type = mRNA
acttgcctggacgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaa

SEQ ID NO: 51 - RNA, 572 nt, Homo sapiens
  source 1..572: mol_type = mRNA
acttgcctggacgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaa

SEQ ID NO: 52 - RNA, 542 nt, Homo sapiens
  source 1..542: mol_type = mRNA
acttgcctggacgctgcgccacatcccaccggcccttacactgtggtgtccagcagcatccggcttcatggggggacttgaaccctgcagcaggctcctgctcctgcctctcctgctggctgtaagtgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaa

SEQ ID NO: 53 - RNA, 539 nt, Homo sapiens
  source 1..539: mol_type = mRNA
acttgcctggacgctgcgccacatcccaccggcccttacactgtggtgtccagcagcatccggcttcatggggggacttgaaccctgcagcaggctcctgctcctgcctctcctgctggctgtaagtgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaa

SEQ ID NO: 54 - RNA, 604 nt, Homo sapiens
  source 1..604: mol_type = mRNA
ccacgcgtccgcgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaaaaaaaaaaaaaaaaaaaaaaaaaaaaaaa

SEQ ID NO: 55 - RNA, 576 nt, Homo sapiens
  source 1..576: mol_type = mRNA
cgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctggagcccaccctgcacctcattccaactcctaccgcgatacagacccacagagtgccatccctgagagaccagaccgctccccaatactctcctaaaataaacatgaagcacaaaaaaaaaaaaaaaaa

SEQ ID NO: 56 - RNA, 561 nt, Homo sapiens
  source 1..561: mol_type = mRNA
acttgcctggacgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatg

SEQ ID NO: 57 - RNA, 350 nt, Homo sapiens
  source 1..350: mol_type = mRNA
catggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatca

SEQ ID NO: 58 - RNA, 342 nt, Homo sapiens
  source 1..342: mol_type = mRNA
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatga

SEQ ID NO: 59 - RNA, 339 nt, Homo sapiens
  source 1..339: mol_type = mRNA
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 60 - RNA, 570 nt, Homo sapiens
  source 1..570: mol_type = mRNA
acttgcctggacgctgcgccacatcccaccggcccttacactgtggtgtccagcagcatccggcttcatggggggacttgaaccctgcagcaggctcctgctcctgcctctcctgctggctgtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaaca

SEQ ID NO: 61 - RNA, 568 nt, Homo sapiens
  source 1..568: mol_type = mRNA
acttgcctggacgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagc

SEQ ID NO: 62 - RNA, 522 nt, Homo sapiens
  source 1..522: mol_type = mRNA
acttgcctggacgctgcgccacatcccaccggcccttacactgtggtgtccagcagcatccggcttcatggggggacttgaaccctgcagcaggctcctgctcctgcctctcctgctggctgtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccaga

SEQ ID NO: 63 - DNA, 541 bp, Homo sapiens
  source 1..541: mol_type = other DNA
acttgcctggacgctgcgccacatcccaccggcccttacactgtggtgtccagcagcatccggcttcatggggggacttgaaccctgcagcaggctcctgctcctgcctctcctgctggctgtaagtgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaaca

SEQ ID NO: 64 - DNA, 415 bp, Homo sapiens
  source 1..415: mol_type = other DNA
acttgcctggacgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatc

SEQ ID NO: 65 - DNA, 620 bp, Homo sapiens
  source 1..620: mol_type = other DNA
tccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggggatggtaatggaggtgctgatttggaatgacaaaacaccctcaactcaccactctgcctgtctctgcaccccacaacacccctcacctccagcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaa

SEQ ID NO: 66 - DNA, 522 bp, Homo sapiens
  source 1..522: mol_type = other DNA
acatcccaccggcccttacactgtggtgtccagcagcatccggcttcatggggggacttgaaccctgcagcaggctcctgctcctgcctctcctgctggctgtaagtgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaa

SEQ ID NO: 67 - DNA, 575 bp, Homo sapiens
  source 1..575: mol_type = other DNA
acttgcctggacgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaa

SEQ ID NO: 68 - DNA, 572 bp, Homo sapiens
  source 1..572: mol_type = other DNA
acttgcctggacgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaa

SEQ ID NO: 69 - DNA, 542 bp, Homo sapiens
  source 1..542: mol_type = other DNA
acttgcctggacgctgcgccacatcccaccggcccttacactgtggtgtccagcagcatccggcttcatggggggacttgaaccctgcagcaggctcctgctcctgcctctcctgctggctgtaagtgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaa

SEQ ID NO: 70 - DNA, 539 bp, Homo sapiens
  source 1..539: mol_type = other DNA
acttgcctggacgctgcgccacatcccaccggcccttacactgtggtgtccagcagcatccggcttcatggggggacttgaaccctgcagcaggctcctgctcctgcctctcctgctggctgtaagtgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaa

SEQ ID NO: 71 - DNA, 604 bp, Homo sapiens
  source 1..604: mol_type = other DNA
ccacgcgtccgcgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaaaaaaaaaaaaaaaaaaaaaaaaaaaaaaa

SEQ ID NO: 72 - DNA, 576 bp, Homo sapiens
  source 1..576: mol_type = other DNA
cgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctggagcccaccctgcacctcattccaactcctaccgcgatacagacccacagagtgccatccctgagagaccagaccgctccccaatactctcctaaaataaacatgaagcacaaaaaaaaaaaaaaaaa

SEQ ID NO: 73 - DNA, 561 bp, Homo sapiens
  source 1..561: mol_type = other DNA
acttgcctggacgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatg

SEQ ID NO: 74 - DNA, 350 bp, Homo sapiens
  source 1..350: mol_type = other DNA
catggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatca

SEQ ID NO: 75 - DNA, 342 bp, Homo sapiens
  source 1..342: mol_type = other DNA
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatga

SEQ ID NO: 76 - DNA, 339 bp, Homo sapiens
  source 1..339: mol_type = other DNA
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 77 - DNA, 570 bp, Homo sapiens
  source 1..570: mol_type = other DNA
acttgcctggacgctgcgccacatcccaccggcccttacactgtggtgtccagcagcatccggcttcatggggggacttgaaccctgcagcaggctcctgctcctgcctctcctgctggctgtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaaca

SEQ ID NO: 78 - DNA, 568 bp, Homo sapiens
  source 1..568: mol_type = other DNA
acttgcctggacgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagc

SEQ ID NO: 79 - DNA, 522 bp, Homo sapiens
  source 1..522: mol_type = other DNA
acttgcctggacgctgcgccacatcccaccggcccttacactgtggtgtccagcagcatccggcttcatggggggacttgaaccctgcagcaggctcctgctcctgcctctcctgctggctgtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccaga

SEQ ID NO: 80 - protein, 101 aa, Homo sapiens
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 81 - protein, 112 aa, Homo sapiens
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 82 - protein, 144 aa, Homo sapiens
MGGLEPCSRLLLLPLLLAVSGLRPVQAQAQSDCSCSTVSPGVLAGIVMGDLVLTVLIALAVYFLGRLVPRGRGAAEGMVMEVLIWNDKTPSTHHSACLCTPQHPSPPAATRKQRITETESPYQELQGQRSDVYSDLNTQRPYYK

SEQ ID NO: 83 - protein, 102 aa, Homo sapiens
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 84 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 85 - protein, 35 aa, Homo sapiens
MGGLEPCSRLLLLPLLLAVSVLSRPRPRAIAVALR

(19) United States
(12) Patent Application Publication
(10) Pub. No.: US 2023/0287422 A1
(43) Pub. Date: Sep. 14, 2023

(54) Triggering Receptor Expressed On Myeloid Cells 2 (TREM2) Variants And Uses Thereof

(71) Applicant: Regeneron Pharmaceuticals, Inc., Tarrytown, NY (US)

(72) Inventors: Jan Freudenberg, Tarrytown, NY (US); Giovanni Coppola, Tarrytown, NY (US); Katherine Siminovitch, Tarrytown, NY (US)

(21) Appl. No.: 18/152,346

(22) Filed: Jan. 10, 2023

Related U.S. Application Data
(60) Provisional application No. 63/298,417, filed on Jan. 11, 2022.

(51) Int. Cl. C12N 15/113; C12Q 1/6883

(52) U.S. Cl. CPC C12N 15/113; C12Q 1/6883; C12Q 2600/156

(57) ABSTRACT

The present disclosure provides methods of treating subjects having Alzheimer's Disease, and methods of identifying subjects having an increased risk of developing Alzheimer's Disease.

### REFERENCE TO SEQUENCE LISTING

This application includes a Sequence Listing submitted electronically as an XML file named 381203666SEQ, created on Jan. 4, 2023, with a size of 137 kilobytes. The Sequence Listing is incorporated herein by reference.

### FIELD

The present disclosure relates generally to the treatment of subjects having Alzheimer's Disease and methods of identifying subjects having an increased risk of developing Alzheimer's Disease.

### BACKGROUND

With the increase in life span globally, Alzheimer's disease (AD) featuring memory loss and dementia, poses a more serious problem for the society than ever. Despite an intensive search for therapeutic intervention, there is no drug that has proven completely effective in combating this devastating illness of the nervous system. The cause of AD is virtually unknown, although age is clearly the major risk factor. The pathology that is universal to all AD patients includes severe brain atrophy, neuronal loss, neurofibrillary tangles, and senile plaques composed of aggregated β-amyloid (Aβ) peptides. The pathogenesis of AD involves a heterogeneous mixture of Aβ peptides and amyloid plaque formation in the brain. Sequential cleavage of the amyloid precursor protein (APP) by 8-secretase (BACE1) and the γ-secretase complex results in the generation of Aβ species of varying lengths, e.g. Aβ38, Aβ40, and Aβ42. More neurotoxic than Aβ38 or Aβ40, the Aβ42 peptide is prone to form aggregated amyloid oligomers (i.e., precursor to larger fibrils), which are thought to contribute to plaque formation and cognitive decline.

Triggering Receptor Expressed On Myeloid Cells 2 (TREM2) is a transmembrane signaling receptor that is predominantly expressed on myeloid cells. The ligands of TREM2 encompass anionic molecules, including bacterial products such as LPS, DNA, lipoproteins, and phospholipids. The medical relevance of TREM2 was first recognized when rare homozygous mutations were found to cause autosomal recessive polycystic lipomembranous osteodysplasia with sclerosing leukoencephalopathy (PLOSL), also known as Nasu-Hakola disease. Lipomembranous osteodysplasia is a specific form of fat tissue necrosis that displays bone cysts filled with triglycerides and lamellar structures, which embed crystals and collagenous tissue. PLOSL patients further present with early-onset dementia that is accompanied by cortical and central atrophy, accentuated in the frontal lobes, and occasionally cerebellar atrophy.

Transmembrane Immune Signaling Adaptor (TYROBP) is a transmembrane which contains an immunoreceptor tyrosine-based activation motif (ITAM) in its cytoplasmic domain. TYROBP acts as an adapter protein which non-covalently associates with activating receptors found on the surface of a variety of immune cells to mediate signaling and cell activation following ligand binding by the receptors. In addition, TYROBP associates with TREM2 on monocyte-derived dendritic cells to mediate up-regulation of chemokine receptor CCR7 and dendritic cell maturation and survival. The association with TREM2 mediates cytokine-induced formation of multinucleated giant cells which are formed by the fusion of macrophages. Moreover, TYROBP stabilizes the TREM2 C-terminal fragment (TREM2-CTF) produced by TREM2 ectodomain shedding which suppresses the release of pro-inflammatory cytokines. In microglia, TYROBP is required with TREM2 for phagocytosis of apoptotic neurons

### SUMMARY

The present disclosure provides methods of treating a subject having Alzheimer's Disease or at risk of developing Alzheimer's Disease, the methods comprising administering a TREM2 agonist to the subject.

The present disclosure provides methods of treating a subject with a therapeutic agent that treats or inhibits Alzheimer's Disease, wherein the subject has Alzheimer's Disease or is at risk of developing Alzheimer's Disease, the methods comprising: determining whether the subject has a TREM2 variant nucleic acid molecule by: obtaining or having obtained a biological sample from the subject; and performing or having performed a sequence analysis on the biological sample to determine if the subject has a genotype comprising the TREM2 variant nucleic acid molecule; and when the subject is TREM2 reference, then administering or continuing to administer to the subject the therapeutic agent that treats or inhibits Alzheimer's Disease in a standard dosage amount and/or a TREM2 agonist; and when the subject is heterozygous or homozygous for the TREM2 variant nucleic acid molecule, then administering or continuing to administer to the subject the therapeutic agent that treats or inhibits Alzheimer's Disease in an amount that is the same as or greater than a standard dosage amount and/or a TREM2 agonist; wherein the presence of a genotype having the TREM2 variant nucleic acid molecule indicates the subject has an increased risk of developing Alzheimer's Disease or developing a more severe form of Alzheimer's Disease.

The present disclosure also provides methods of identifying a subject having an increased risk for developing Alzheimer's Disease or a more severe form of Alzheimer's Disease, the method comprising: determining or having determined the presence or absence of a TREM2 variant nucleic acid molecule in a biological sample obtained from the subject; wherein: when the subject is TREM2 reference, then the subject does not have an increased risk for developing Alzheimer's Disease; and when the subject is heterozygous or homozygous for the TREM2 variant nucleic acid molecule, then the subject has an increased risk for developing Alzheimer's Disease or a more severe form of Alzheimer's Disease.

The present disclosure also provides TREM2 agonists for use in the treatment of Alzheimer's Disease.

The present disclosure also provides therapeutic agents that treat or inhibit Alzheimer's Disease for use in the treatment of Alzheimer's Disease in a subject having: a TREM2 variant genomic nucleic acid molecule having a nucleotide sequence comprising a thymine at a position corresponding to position 1,630 according to SEQ ID NO:2, or the complement thereof; a TREM2 variant mRNA molecule having a nucleotide sequence comprising: a uracil at a position corresponding to position 201 according to SEQ ID NO:10, or the complement thereof; a uracil at a position corresponding to position 118 according to SEQ ID NO:11, or the complement thereof; a uracil at a position corresponding to position 131 according to SEQ ID NO:14, or the complement thereof; a uracil at a position corresponding to position 131 according to SEQ ID NO:15, or the complement thereof; a uracil at a position corresponding to position 118 according to SEQ ID NO:16, or the complement thereof; a uracil at a position corresponding to position 201 according to SEQ ID NO:17, or the complement thereof; a uracil at a position corresponding to position 201 according to SEQ ID NO:18, or the complement thereof; a uracil at a position corresponding to position 174 according to SEQ ID NO:19, or the complement thereof; or a uracil at a position corresponding to position 191 according to SEQ ID NO:20 or the complement thereof; or a TREM2 variant cDNA molecule having a nucleotide sequence comprising: a thymine at a position corresponding to position 201 according to SEQ ID NO:30, or the complement thereof; a thymine at a position corresponding to position 118 according to SEQ ID NO:31, or the complement thereof; a thymine at a position corresponding to position 131 according to SEQ ID NO:32, or the complement thereof; a thymine at a position corresponding to position 131 according to SEQ ID NO:33, or the complement thereof; a thymine at a position corresponding to position 118 according to SEQ ID NO:34, or the complement thereof; a thymine at a position corresponding to position 201 according to SEQ ID NO:35, or the complement thereof; a thymine at a position corresponding to position 201 according to SEQ ID NO:36, or the complement thereof; a thymine at a position corresponding to position 174 according to SEQ ID NO:37, or the complement thereof; or a thymine at a position corresponding to position 191 according to SEQ ID NO:38, or the complement thereof.

The present disclosure also provides methods of treating a subject with a therapeutic agent that treats or inhibits Alzheimer's Disease, wherein the subject has Alzheimer's Disease or is at risk of developing Alzheimer's Disease, the methods comprising: determining whether the subject has a TYROBP variant nucleic acid molecule by: obtaining or having obtained a biological sample from the subject; and performing or having performed a sequence analysis on the biological sample to determine if the subject has a genotype comprising the TYROBP variant nucleic acid molecule; and when the subject is TYROBP reference, then administering or continuing to administer to the subject the therapeutic agent that treats or inhibits Alzheimer's Disease in a standard dosage amount; and when the subject is heterozygous or homozygous for the TYROBP variant nucleic acid molecule, then administering or continuing to administer to the subject the therapeutic agent that treats or inhibits Alzheimer's Disease in an amount that is the same as or greater than a standard dosage amount; wherein the presence of a genotype having the TYROBP variant nucleic acid molecule indicates the subject has an increased risk of developing Alzheimer's Disease or developing a more severe form of Alzheimer's Disease.

The present disclosure also provides methods of identifying a subject having an increased risk for developing Alzheimer's Disease or a more severe form of Alzheimer's Disease, the methods comprising: determining or having determined the presence or absence of a TYROBP variant nucleic acid molecule in a biological sample obtained from the subject; wherein: when the subject is TYROBP reference, then the subject does not have an increased risk for developing Alzheimer's Disease; and when the subject is heterozygous or homozygous for a TYROBP variant nucleic acid molecule, then the subject has an increased risk for developing Alzheimer's Disease or a more severe form of Alzheimer's Disease.

The present disclosure also provides therapeutic agents that treat or inhibit Alzheimer's Disease for use in the treatment of Alzheimer's Disease in a subject having: a TYROBP genomic nucleic acid molecule having a nucleotide sequence comprising any one or more of: 19:35907523:AG:A, 19:35907461:G:A, 19:35907250:G:A, 19:35907729:C:T, 19:35908220:TC:T, 19:35907582:T:C, 19:35908220:T:TC, 19:35907248:CTG:C, 19:35907539:TCC:T, 19:35904569:T:G, 19:35904575:G:C, 19:35904575:G:T, 19:35904622:G:A, 19:35904635:C:T, 19:35907222:TA:T, 19:35907247:G:GCTGTTTCC, 19:35908208:G:T, or 19:35908228:T:C; an TYROBP mRNA molecule having a nucleotide sequence comprising any one or more of: 19:35907523:AG:A, 19:35907461:G:A, 19:35907250:G:A, 19:35907729:C:T, 19:35908220:TC:T, 19:35907582:T:C, 19:35908220:T:TC, 19:35907248:CTG:C, 19:35907539:TCC:T, 19:35904569:T:G, 19:35904575:G:C, 19:35904575:G:T, 19:35904622:G:A, 19:35904635:C:T, 19:35907222:TA:T, 19:35907247:G:GCTGTTTCC, 19:35908208:G:T, or 19:35908228:T:C; or a TYROBP cDNA molecule having a nucleotide sequence comprising any one or more of: 19:35907523:AG:A, 19:35907461:G:A, 19:35907250:G:A, 19:35907729:C:T, 19:35908220:TC:T, 19:35907582:T:C, 19:35908220:T:TC, 19:35907248:CTG:C, 19:35907539:TCC:T, 19:35904569:T:G, 19:35904575:G:C, 19:35904575:G:T, 19:35904622:G:A, 19:35904635:C:T, 19:35907222:TA:T, 19:35907247:G:GCTGTTTCC, 19:35908208:G:T, or 19:35908228:T:C.

### BRIEF DESCRIPTION OF THE DRAWINGS

The accompanying FIGURES, which are incorporated in and constitute a part of this specification, illustrate several features of the present disclosure.

FIG. 1 shows distribution of TREM2 pLoF variants across the gene and its different splice isoform; three different isoforms exist that differ in their C-terminal sequence; the two most frequent pLoFs (Trp191X and c.483-splice) do not affect the longest canonical transcript (L-form); the third pLoF (Q33X), which has a substantially lower allele frequency and was previously reported cause PLOSL when homozygote, affects all three transcripts including the L-form; this transcript is thought to be membrane bound and mediate TREM2 signaling on microglial cells; sequences (from top to bottom)=amino acids 1-161 of SEQ ID NO:39; amino acids 162-230 of SEQ ID NO:41; amino acids 162-222 of SEQ ID NO:40; and amino acids 192-249 of SEQ ID NO:42.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

Rare loss-of-function variants in the TREM2 gene associated with an increased risk of developing Alzheimer's Disease in subjects has been identified in accordance with the present disclosure. For example, a genetic alteration that changes the cytosine nucleotide of position 1,630 in the human TREM2 reference (see, SEQ ID NO:1) to thymine, has been observed to indicate that the human having such an alteration may have an increased risk of developing Alzheimer's Disease. It is believed that no loss-of-function variants of the TREM2 gene or protein have any known association with Alzheimer's Disease. Altogether, the genetic analyses described herein surprisingly indicate that the TREM2 gene and, in particular, loss-of-function variants in the TREM2 gene, associate with an increased risk of developing Alzheimer's Disease. Therefore, subjects that have a TREM2 variant nucleic acid molecule or polypeptide that associates with an increased risk of developing Alzheimer's Disease may be treated such that the Alzheimer's Disease is prevented, the symptoms thereof are reduced, and/or development of symptoms is repressed. Accordingly, the present disclosure provides methods of leveraging the identification of such variants in subjects to identify or stratify risk in such subjects of developing Alzheimer's Disease, or to diagnose subjects as having an increased risk of developing Alzheimer's Disease or a more severe form of Alzheimer's Disease, such that subjects at risk or subjects with active disease may be treated accordingly.

For purposes of the present disclosure, any particular human can be categorized as having one of three TREM2 genotypes: i) TREM2 reference; ii) heterozygous for a TREM2 variant nucleic acid molecule; or iii) homozygous for a TREM2 variant nucleic acid molecule. A human is TREM2 reference when the human does not have a copy of a TREM2 variant nucleic acid molecule. A human is heterozygous for a TREM2 variant nucleic acid molecule when the human has a single copy of a TREM2 variant nucleic acid molecule. A human is homozygous for a TREM2 variant nucleic acid molecule when the human has two copies of a TREM2 variant nucleic acid molecule. A human who has a TREM2 polypeptide having a partial loss-of-function (or predicted partial loss-of-function) is hypomorphic for TREM2.

As used herein, a TREM2 variant nucleic acid molecule is any TREM2 nucleic acid molecule (such as, a genomic nucleic acid molecule, an mRNA molecule, or a cDNA molecule) encoding a TREM2 polypeptide having a partial loss-of-function, a complete loss-of-function, a predicted partial loss-of-function, or a predicted complete loss-of-function. The TREM2 variant nucleic acid molecule can also be a missense variant, a splice-site variant, a stop-gain variant, a start-loss variant, a stop-loss variant, a frameshift variant, or an in-frame indel variant, or a variant that encodes a truncated TREM2 predicted loss-of-function polypeptide. A TREM2 variant nucleic acid molecule can also be any nucleic acid molecule (such as, a genomic nucleic acid molecule, an mRNA molecule, or a cDNA molecule) resulting in complete loss or decreased or aberrant expression of TREM2 mRNA or polypeptide. A TREM2 variant nucleic acid molecule can also be any missense variant nucleic acid molecule (such as, a genomic nucleic acid molecule, an mRNA molecule, or a cDNA molecule) resulting in a reduction of TREM2 activity. A TREM2 variant nucleic acid molecule can also be any TREM2 nucleic acid molecule that results in a reduction of TREM2/TYROBP signaling. A TREM2 variant nucleic acid molecule can also be any TREM2 nucleic acid molecule that alters, such as by reducing, the L-form of mRNA expression level.

In any of the embodiments described herein, the TREM2 variant nucleic acid molecule can be any nucleic acid molecule encoding TREM2 Gln33STOP or Gln63STOP. In some embodiments, the TREM2 variant nucleic acid molecule encodes TREM2 Gln33STOP. In some embodiments, the TREM2 variant nucleic acid molecule encodes TREM2 Gln63STOP. In any of the embodiments described herein, the TREM2 predicted loss-of-function polypeptide can be any TREM2 polypeptide having a partial loss-of-function, a complete loss-of-function, a predicted partial loss-of-function, or a predicted complete loss-of-function. In any of the embodiments described herein, the TREM2 predicted loss-of-function polypeptide can be any of the TREM2 polypeptides described herein including, for example, TREM2 Gln33STOP or Gln63STOP. In some embodiments, the TREM2 predicted loss-of-function variant polypeptide is TREM2 Gln33STOP or Gln63STOP. In some embodiments, the TREM2 predicted loss-of-function variant polypeptide is TREM2 Gln33STOP. In some embodiments, the TREM2 predicted loss-of-function variant polypeptide is TREM2 Gln63STOP.

For subjects or subjects that are genotyped or determined to be heterozygous or homozygous for a TREM2 variant nucleic acid molecule, such subjects or subjects have an increased risk of developing Alzheimer's Disease, such as early-onset Alzheimer's Disease, late-onset Alzheimer's Disease, and familial Alzheimer's Disease. For subjects or subjects that are genotyped or determined to be heterozygous or homozygous for a TREM2 variant nucleic acid molecule, such subjects or subjects can be treated with an agent effective to treat a Alzheimer's Disease, such as early-onset Alzheimer's Disease, late-onset Alzheimer's Disease, and familial Alzheimer's Disease.

Rare loss-of-function variants in the TYROBP gene associated with an increased risk of developing Alzheimer's Disease in subjects has been identified in accordance with the present disclosure. It is believed that no variants of the TYROBP gene or protein have any known association with Alzheimer's Disease. Altogether, the genetic analyses described herein indicate that the TYROBP gene and, in particular, variants in the TYROBP gene, cause an increased risk of developing Alzheimer's Disease. Therefore, subjects that have a TYROBP variant nucleic acid molecule or polypeptide that associates with an increased risk of developing Alzheimer's Disease may be treated such that the Alzheimer's Disease is prevented, the symptoms thereof are reduced, and/or development of symptoms is repressed. Accordingly, the present disclosure provides methods of leveraging the identification of such variants in subjects to identify or stratify risk in such subjects of developing Alzheimer's Disease, or to diagnose subjects as having an increased risk of developing Alzheimer's Disease or a more severe form of Alzheimer's Disease, such that subjects at risk or subjects with active disease may be treated accordingly.

For purposes of the present disclosure, any particular human can be categorized as having one of three TYROBP genotypes: i) TYROBP reference; ii) heterozygous for a TYROBP variant nucleic acid molecule; or iii) homozygous for a TYROBP variant nucleic acid molecule. A human is TYROBP reference when the human does not have a copy of a TYROBP variant nucleic acid molecule. A human is heterozygous for a TYROBP variant nucleic acid molecule when the human has a single copy of a TYROBP variant nucleic acid molecule. A human is homozygous for a TYROBP variant nucleic acid molecule when the human has two copies of a TYROBP variant nucleic acid molecule. A human who has a TYROBP polypeptide having a partial loss-of-function (or predicted partial loss-of-function) is hypomorphic for TYROBP.

As used herein, a TYROBP variant nucleic acid molecule is any TYROBP nucleic acid molecule (such as, a genomic nucleic acid molecule, an mRNA molecule, or a cDNA molecule) encoding a TYROBP polypeptide having a partial loss-of-function, a complete loss-of-function, a predicted partial loss-of-function, or a predicted complete loss-of-function. The TYROBP variant nucleic acid molecule can also be a missense variant, a splice-site variant, a stop-gain variant, a start-loss variant, a stop-loss variant, a frameshift variant, or an in-frame indel variant, or a variant that encodes a truncated TYROBP predicted loss-of-function polypeptide. A TYROBP variant nucleic acid molecule can also be any nucleic acid molecule (such as, a genomic nucleic acid molecule, an mRNA molecule, or a cDNA molecule) resulting in complete loss or decreased or aberrant expression of TYROBP mRNA or polypeptide. A TYROBP variant nucleic acid molecule can also be any missense variant nucleic acid molecule (such as, a genomic nucleic acid molecule, an mRNA molecule, or a cDNA molecule) resulting in a reduction of TYROBP activity. A TYROBP variant nucleic acid molecule can also be any TYROBP nucleic acid molecule that results in a reduction of TREM2/TYROBP signaling.

For subjects or subjects that are genotyped or determined to be heterozygous or homozygous for a TYROBP variant nucleic acid molecule, such subjects or subjects have an increased risk of developing Alzheimer's Disease, such as early-onset Alzheimer's Disease, late-onset Alzheimer's Disease, and familial Alzheimer's Disease. For subjects or subjects that are genotyped or determined to be heterozygous or homozygous for a TYROBP variant nucleic acid molecule, such subjects or subjects can be treated with an agent effective to treat a Alzheimer's Disease, such as early-onset Alzheimer's Disease, late-onset Alzheimer's Disease, and familial Alzheimer's Disease.

In any of the embodiments described herein, Alzheimer's Disease is early-onset Alzheimer's Disease, late-onset Alzheimer's Disease, or familial Alzheimer's Disease. In any of the embodiments described herein, Alzheimer's Disease is early-onset Alzheimer's Disease. In any of the embodiments described herein, Alzheimer's Disease is late-onset Alzheimer's Disease. In any of the embodiments described herein, Alzheimer's Disease is familial Alzheimer's Disease.

Symptoms of mild Alzheimer's Disease include, but are not limited to, memory loss, poor judgment leading to bad decisions, loss of spontaneity and sense of initiative, taking longer to complete normal daily tasks, repeating questions, trouble handling money and paying bills, wandering and getting lost, losing things or misplacing them in odd places, mood and personality changes, increased anxiety and/or aggression. Symptoms of moderate Alzheimer's Disease include, but are not limited to, increased memory loss and confusion, inability to learn new things, difficulty with language and problems with reading, writing, and working with numbers, difficulty organizing thoughts and thinking logically, shortened attention span, problems coping with new situations, difficulty carrying out multistep tasks, such as getting dressed, problems recognizing family and friends, hallucinations, delusions, and paranoia, impulsive behavior such as undressing at inappropriate times or places or using vulgar language, inappropriate outbursts of anger, restlessness, agitation, anxiety, tearfulness, wandering-especially in the late afternoon or evening, repetitive statements or movement, occasional muscle twitches. Symptoms of moderate Alzheimer's Disease include, but are not limited to, inability to communicate, weight loss, seizures, skin infections, difficulty swallowing, groaning, moaning, or grunting, increased sleeping, loss of bowel and bladder control.

The present disclosure provides methods of treating a subject with a therapeutic agent that treats or inhibits Alzheimer's Disease. In some embodiments, the subject has Alzheimer's Disease. In some embodiments, the subject has an increased risk of developing Alzheimer's Disease. In some embodiments, the methods comprise administering or continuing to administer to the subject a TREM2 agonist.

The present disclosure also provides methods of treating a subject with a therapeutic agent that treats or inhibits Alzheimer's Disease. In some embodiments, the subject has Alzheimer's Disease. In some embodiments, the subject has an increased risk of developing Alzheimer's Disease. In some embodiments, the methods comprise determining whether the subject has a TREM2 variant nucleic acid molecule by obtaining or having obtained a biological sample from the subject, and performing or having performed a sequence analysis on the biological sample to determine if the subject has a genotype comprising the TREM2 variant nucleic acid molecule. When the subject is TREM2 reference, the therapeutic agent that treats or inhibits Alzheimer's Disease is administered or continued to be administered to the subject in a standard dosage amount and/or a TREM2 agonist. In some embodiments when the subject is TREM2 reference, a TREM2 agonist is administered or continued to be administered to the subject. When the subject is heterozygous or homozygous for the TREM2 variant nucleic acid molecule, the therapeutic agent that treats or inhibits Alzheimer's Disease is administered or continued to be administered to the subject in an amount that is the same as or greater than a standard dosage amount and/or a TREM2 agonist. In some embodiments when the subject is heterozygous or homozygous for the TREM2 variant nucleic acid molecule, the TREM2 agonist is administered or continued to be administered to the subject. The presence of a genotype having the TREM2 variant nucleic acid molecule indicates the subject has an increased risk of developing Alzheimer's Disease, or a more severe form of Alzheimer's Disease. In some embodiments, the subject is TREM2 reference. In some embodiments, the subject is heterozygous for a TREM2 variant nucleic acid molecule. In some embodiments, the subject is homozygous for a TREM2 variant nucleic acid molecule.

In some embodiments, the methods of treatment further comprise detecting the presence or absence of a TREM2 variant nucleic acid molecule in a biological sample from the subject. Detecting the presence or absence of a TREM2 variant nucleic acid molecule in a biological sample from a subject and/or determining whether a subject has a TREM2 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

The present disclosure also provides methods of treating a subject with a therapeutic agent that treats or inhibits Alzheimer's Disease. In some embodiments, the subject is suffering from Alzheimer's Disease. In some embodiments, the method comprises determining whether the subject has a TREM2 predicted loss-of-function polypeptide by obtaining or having obtained a biological sample from the subject, and performing or having performed an assay on the biological sample to determine if the subject has a TREM2 predicted loss-of-function polypeptide. When the subject does not have a TREM2 predicted loss-of-function polypeptide, the therapeutic agent that treats or inhibits Alzheimer's Disease is administered or continued to be administered to the subject in a standard dosage amount. When the subject has a TREM2 predicted loss-of-function polypeptide, the therapeutic agent that treats or inhibits Alzheimer's Disease is administered or continued to be administered to the subject in an amount that is the same as or greater than a standard dosage amount. The presence of a TREM2 predicted loss-of-function polypeptide indicates the subject has an increased risk of developing Alzheimer's Disease, or a more severe form of Alzheimer's Disease. In some embodiments, the subject has a TREM2 predicted loss-of-function polypeptide. In some embodiments, the subject does not have a TREM2 predicted loss-of-function polypeptide. In some embodiments, the therapeutic agent that treats or inhibits Alzheimer's Disease is a TREM2 agonist.

Detecting the presence or absence of a TREM2 predicted loss-of-function polypeptide in a biological sample from a subject and/or determining whether a subject has a TREM2 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

The present disclosure provides methods of treating a subject with a therapeutic agent that treats or inhibits Alzheimer's Disease. In some embodiments, the subject has Alzheimer's Disease. In some embodiments, the subject has an increased risk of developing Alzheimer's Disease. In some embodiments, the methods comprise administering or continuing to administer to the subject a TYROBP agonist.

The present disclosure also provides methods of treating a subject with a therapeutic agent that treats or inhibits Alzheimer's Disease. In some embodiments, the subject has Alzheimer's Disease. In some embodiments, the subject has an increased risk of developing Alzheimer's Disease. In some embodiments, the methods comprise determining whether the subject has a TYROBP variant nucleic acid molecule by obtaining or having obtained a biological sample from the subject, and performing or having performed a sequence analysis on the biological sample to determine if the subject has a genotype comprising the TYROBP variant nucleic acid molecule. When the subject is TYROBP reference, the therapeutic agent that treats or inhibits Alzheimer's Disease is administered or continued to be administered to the subject in a standard dosage amount. When the subject is heterozygous or homozygous for the TYROBP variant nucleic acid molecule, the therapeutic agent that treats or inhibits Alzheimer's Disease is administered or continued to be administered to the subject in an amount that is the same as or greater than a standard dosage amount. The presence of a genotype having the TYROBP variant nucleic acid molecule indicates the subject has an increased risk of developing Alzheimer's Disease, or a more severe form of Alzheimer's Disease. In some embodiments, the subject is TYROBP reference. In some embodiments, the subject is heterozygous for a TYROBP variant nucleic acid molecule. In some embodiments, the subject is homozygous for a TYROBP variant nucleic acid molecule.

In some embodiments, the methods of treatment further comprise detecting the presence or absence of a TYROBP variant nucleic acid molecule in a biological sample from the subject. Detecting the presence or absence of a TYROBP variant nucleic acid molecule in a biological sample from a subject and/or determining whether a subject has a TYROBP variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

The present disclosure also provides methods of treating a subject with a therapeutic agent that treats or inhibits Alzheimer's Disease. In some embodiments, the subject is suffering from Alzheimer's Disease. In some embodiments, the method comprises determining whether the subject has a TYROBP predicted loss-of-function polypeptide by obtaining or having obtained a biological sample from the subject, and performing or having performed an assay on the biological sample to determine if the subject has a TYROBP predicted loss-of-function polypeptide. When the subject does not have a TYROBP predicted loss-of-function polypeptide, the therapeutic agent that treats or inhibits Alzheimer's Disease is administered or continued to be administered to the subject in a standard dosage amount. When the subject has a TYROBP predicted loss-of-function polypeptide, the therapeutic agent that treats or inhibits Alzheimer's Disease is administered or continued to be administered to the subject in an amount that is the same as or greater than a standard dosage amount. The presence of a TYROBP predicted loss-of-function polypeptide indicates the subject has an increased risk of developing Alzheimer's Disease, or a more severe form of Alzheimer's Disease. In some embodiments, the subject has a TYROBP predicted loss-of-function polypeptide. In some embodiments, the subject does not have a TYROBP predicted loss-of-function polypeptide.

Detecting the presence or absence of a TYROBP predicted loss-of-function polypeptide in a biological sample from a subject and/or determining whether a subject has a TYROBP predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

Examples of therapeutic agents that treat or inhibit Alzheimer's Disease include, but are not limited to, mentamine, rivastigmine, galantamine, donepezil, and aducanumab. In some embodiments, the therapeutic agent that treats or inhibits Alzheimer's Disease is mentamine. In some embodiments, the therapeutic agent that treats or inhibits Alzheimer's Disease is rivastigmine. In some embodiments, the therapeutic agent that treats or inhibits Alzheimer's Disease is galantamine. In some embodiments, the therapeutic agent that treats or inhibits Alzheimer's Disease is donepezil. In some embodiments, the therapeutic agent that treats or inhibits Alzheimer's Disease is aducanumab.

In some embodiments, the therapeutic agent that treats or inhibits Alzheimer's Disease is an RNAi molecule.

In some embodiments, the therapeutic agent that treats or inhibits Alzheimer's Disease is a TREM2 agonist. In some embodiments, the TREM2 agonist is a TREM2 activating antibody or small molecule. In some embodiments, the TREM2 agonist is a TREM2 missense variant that has a functionally activating effect.

In some embodiments, the therapeutic agent that treats or inhibits Alzheimer's Disease is a TYROBP agonist. In some embodiments, the TYROBP agonist is a TREM2 protein. In some embodiments, the TYROBP agonist is a small molecule.

In some embodiments, the dose of the therapeutic agents that treat or inhibit Alzheimer's Disease can be increased by about 10%, by about 20%, by about 30%, by about 40%, by about 50%, by about 60%, by about 70%, by about 80%, or by about 90% for subjects or subjects that are heterozygous or homozygous for a TREM2 variant nucleic acid molecule and/or a TYROBP variant nucleic acid molecule (i.e., a greater amount than the standard dosage amount) compared to subjects or subjects that are TREM2 reference and/or TYROBP reference (who may receive a standard dosage amount). In some embodiments, the dose of the therapeutic agents that treat or inhibit Alzheimer's Disease can be increased by about 10%, by about 20%, by about 30%, by about 40%, or by about 50%. In addition, the dose of therapeutic agents that treat or inhibit Alzheimer's Disease in subjects or subjects that are heterozygous or homozygous for a TREM2 variant nucleic acid molecule and/or a TYROBP variant nucleic acid molecule can be administered more frequently compared to subjects or subjects that are TREM2 reference and/or TYROBP reference.

In some embodiments, the dose of the therapeutic agents that treat or inhibit Alzheimer's Disease can be increased by about 10%, by about 20%, by about 30%, by about 40%, by about 50%, by about 60%, by about 70%, by about 80%, or by about 90% for subjects or subjects that are homozygous for a TREM2 variant nucleic acid molecule and/or a TYROBP variant nucleic acid molecule compared to subjects or subjects that are heterozygous for a TREM2 variant nucleic acid molecule and/or a TYROBP variant nucleic acid molecule. In some embodiments, the dose of the therapeutic agents that treat or inhibit Alzheimer's Disease can be increased by about 10%, by about 20%, by about 30%, by about 40%, or by about 50%. In addition, the dose of therapeutic agents that treat or inhibit Alzheimer's Disease in subjects or subjects that are homozygous for a TREM2 variant nucleic acid molecule and/or a TYROBP variant nucleic acid molecule can be administered more frequently compared to subjects or subjects that are heterozygous for a TREM2 variant nucleic acid molecule and/or a TYROBP variant nucleic acid molecule.

Administration of the therapeutic agents that treat or inhibit Alzheimer's Disease can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that treat or inhibit Alzheimer's Disease can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in Alzheimer's Disease, a decrease/reduction in the severity of Alzheimer's Disease (such as, for example, a reduction or inhibition of development or Alzheimer's Disease), a decrease/reduction in symptoms and Alzheimer's Disease-related effects, delaying the onset of symptoms and Alzheimer's Disease-related effects, reducing the severity of symptoms of Alzheimer's Disease-related effects, reducing the severity of an acute episode, reducing the number of symptoms and Alzheimer's Disease-related effects, reducing the latency of symptoms and Alzheimer's Disease-related effects, an amelioration of symptoms and Alzheimer's Disease-related effects, reducing secondary symptoms, reducing secondary infections, preventing relapse to Alzheimer's Disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of Alzheimer's Disease development/progression (such as, for example, a complete or partial avoidance/inhibition or a delay), and an increased survival time of the affected host animal, following administration of a therapeutic protocol. Treatment of Alzheimer's Disease encompasses the treatment of subjects already diagnosed as having any form of Alzheimer's Disease at any clinical stage or manifestation, the delay of the onset or evolution or aggravation or deterioration of the symptoms or signs of Alzheimer's Disease, and/or preventing and/or reducing the severity of Alzheimer's Disease.

The present disclosure also provides methods of identifying a subject having an increased risk for developing Alzheimer's Disease or a more severe form of Alzheimer's Disease. In some embodiments, the methods comprise determining or having determined in a biological sample obtained from the subject the presence or absence of a TREM2 variant nucleic acid molecule (such as a genomic nucleic acid molecule, mRNA molecule, and/or cDNA molecule). When the subject lacks a TREM2 variant nucleic acid molecule (i.e., the subject is genotypically categorized as a TREM2 reference), then the subject does not have an increased risk for developing Alzheimer's Disease or a more severe form of Alzheimer's Disease. When the subject has a TREM2 variant nucleic acid molecule (i.e., the subject is heterozygous or homozygous for a TREM2 variant nucleic acid molecule), then the subject has an increased risk for developing Alzheimer's Disease or a more severe form of Alzheimer's Disease.

Determining whether a subject has a TREM2 variant nucleic acid molecule in a biological sample from a subject and/or determining whether a subject has a TREM2 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing Alzheimer's Disease or a more severe form of Alzheimer's Disease, the subject is further treated with a therapeutic agent that treats or inhibits Alzheimer's Disease, as described herein. In some embodiments, when the subject is heterozygous or homozygous for a TREM2 variant nucleic acid molecule, the subject is administered the therapeutic agent that treats or inhibits Alzheimer's Disease in a dosage amount that is the same as or greater than a standard dosage amount. In some embodiments, when the subject is homozygous for a TREM2 variant nucleic acid molecule, the subject is administered the therapeutic agent that treats or inhibits Alzheimer's Disease in a dosage amount that is the same as or greater than the dosage amount administered to a subject that is heterozygous for a TREM2 variant nucleic acid molecule. In some embodiments, the subject is TREM2 reference. In some embodiments, the subject is heterozygous for a TREM2 variant nucleic acid molecule. In some embodiments, the subject is homozygous for a TREM2 variant nucleic acid molecule. In some embodiments, the therapeutic agent that treats or inhibits Alzheimer's Disease is a TREM2 agonist.

The present disclosure also provides methods of identifying a subject having an increased risk for developing Alzheimer's Disease or a more severe form of Alzheimer's Disease. In some embodiments, the methods comprise determining or having determined in a biological sample obtained from the subject the presence or absence of a TYROBP variant nucleic acid molecule (such as a genomic nucleic acid molecule, mRNA molecule, and/or cDNA molecule). When the subject lacks a TYROBP variant nucleic acid molecule (i.e., the subject is genotypically categorized as a TYROBP reference), then the subject does not have an increased risk for developing Alzheimer's Disease or a more severe form of Alzheimer's Disease. When the subject has a TYROBP variant nucleic acid molecule (i.e., the subject is heterozygous or homozygous for a TYROBP variant nucleic acid molecule), then the subject has an increased risk for developing Alzheimer's Disease or a more severe form of Alzheimer's Disease.

Determining whether a subject has a TYROBP variant nucleic acid molecule in a biological sample from a subject and/or determining whether a subject has a TYROBP variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing Alzheimer's Disease or a more severe form of Alzheimer's Disease, the subject is further treated with a therapeutic agent that treats or inhibits Alzheimer's Disease, as described herein. In some embodiments, when the subject is heterozygous or homozygous for a TYROBP variant nucleic acid molecule, the subject is administered the therapeutic agent that treats or inhibits Alzheimer's Disease in a dosage amount that is the same as or greater than a standard dosage amount. In some embodiments, when the subject is homozygous for a TYROBP variant nucleic acid molecule, the subject is administered the therapeutic agent that treats or inhibits Alzheimer's Disease in a dosage amount that is the same as or greater than the dosage amount administered to a subject that is heterozygous for a TYROBP variant nucleic acid molecule. In some embodiments, the subject is TYROBP reference. In some embodiments, the subject is heterozygous for a TYROBP variant nucleic acid molecule. In some embodiments, the subject is homozygous for a TYROBP variant nucleic acid molecule.

The present disclosure also provides methods of detecting the presence or absence of a TREM2 variant genomic nucleic acid molecule in a biological sample from a subject, and/or a TREM2 variant mRNA molecule in a biological sample from a subject, and/or a TREM2 variant cDNA molecule produced from an mRNA molecule in a biological sample from a subject. It is understood that gene sequences within a population and mRNA molecules encoded by such genes can vary due to polymorphisms such as single-nucleotide polymorphisms. The sequences provided herein for the TREM2 variant genomic nucleic acid molecule, TREM2 variant mRNA molecule, and TREM2 variant cDNA molecule are only exemplary sequences. Other sequences for the TREM2 variant genomic nucleic acid molecule, variant mRNA molecule, and variant cDNA molecule are also possible.

The present disclosure also provides methods of detecting the presence or absence of a TYROBP variant genomic nucleic acid molecule in a biological sample from a subject, and/or a TYROBP variant mRNA molecule in a biological sample from a subject, and/or a TYROBP variant cDNA molecule produced from an mRNA molecule in a biological sample from a subject. It is understood that gene sequences within a population and mRNA molecules encoded by such genes can vary due to polymorphisms such as single-nucleotide polymorphisms. The sequences provided herein for the TYROBP variant genomic nucleic acid molecule, TYROBP variant mRNA molecule, and TYROBP variant cDNA molecule are only exemplary sequences. Other sequences for the TYROBP variant genomic nucleic acid molecule, variant mRNA molecule, and variant cDNA molecule are also possible.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any TREM2 or TYROBP variant nucleic acid molecule, preliminary processing designed to isolate or enrich the biological sample for the genomic DNA can be employed. A variety of techniques may be used for this purpose. When detecting the level of any TREM2 or TYROBP variant mRNA molecule, different techniques can be used enrich the biological sample with mRNA molecules. Various methods to detect the presence or level of an mRNA molecule or the presence of a particular variant genomic DNA locus can be used.

In some embodiments, detecting a TREM2 or TYROBP variant nucleic acid molecule in a subject comprises carrying out a sequence analysis on a biological sample obtained from the subject to determine whether a TREM2 or TYROBP genomic nucleic acid molecule in the biological sample, and/or a TREM2 or TYROBP mRNA molecule in the biological sample, and/or a TREM2 or TYROBP cDNA molecule produced from an mRNA molecule in the biological sample, comprises one or more variations of any of the variant nucleic acid molecules described herein.

In some embodiments, the methods of detecting the presence or absence of a TREM2 or TYROBP variant nucleic acid molecule (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the nucleotide sequence of TREM2 comprises: a thymine at a position corresponding to position 1,630 according to SEQ ID NO:2 (for genomic nucleic acid molecules); a uracil at a position corresponding to position 201 according to SEQ ID NO:12; a uracil at a position corresponding to position 118 according to SEQ ID NO:13; a uracil at a position corresponding to position 131 according to SEQ ID NO:14; a uracil at a position corresponding to position 131 according to SEQ ID NO:15; a uracil at a position corresponding to position 118 according to SEQ ID NO:16; a uracil at a position corresponding to position 201 according to SEQ ID NO:17; a uracil at a position corresponding to position 201 according to SEQ ID NO:18; a uracil at a position corresponding to position 174 according to SEQ ID NO:19; or a uracil at a position corresponding to position 191 according to SEQ ID NO:20 (for mRNA molecules); a thymine at a position corresponding to position 201 according to SEQ ID NO:30; a thymine at a position corresponding to position 118 according to SEQ ID NO:31; a thymine at a position corresponding to position 131 according to SEQ ID NO:32; a thymine at a position corresponding to position 131 according to SEQ ID NO:33; a thymine at a position corresponding to position 118 according to SEQ ID NO:34; a thymine at a position corresponding to position 201 according to SEQ ID NO:35; a thymine at a position corresponding to position 201 according to SEQ ID NO:36; a thymine at a position corresponding to position 174 according to SEQ ID NO:37; or a thymine at a position corresponding to position 191 according to SEQ ID NO:38 (for CDNA molecules).

In some embodiments, the nucleotide sequence of a TREM2 genomic nucleic acid molecule a thymine at a position corresponding to position 1,630 according to SEQ ID NO:2, or the complement thereof.

In some embodiments, the nucleotide sequence of a TREM2 mRNA molecule comprises: a uracil at a position corresponding to position 201 according to SEQ ID NO:12, or the complement thereof; a uracil at a position corresponding to position 118 according to SEQ ID NO:13, or the complement thereof; a uracil at a position corresponding to position 131 according to SEQ ID NO:14, or the complement thereof; a uracil at a position corresponding to position 131 according to SEQ ID NO:15, or the complement thereof; a uracil at a position corresponding to position 118 according to SEQ ID NO:16, or the complement thereof; a uracil at a position corresponding to position 201 according to SEQ ID NO:17, or the complement thereof; a uracil at a position corresponding to position 201 according to SEQ ID NO:18, or the complement thereof; a uracil at a position corresponding to position 174 according to SEQ ID NO:19, or the complement thereof; or a uracil at a position corresponding to position 191 according to SEQ ID NO:20 or the complement thereof.

In some embodiments, the nucleotide sequence of a TREM2 cDNA molecule comprises: a thymine at a position corresponding to position 201 according to SEQ ID NO:30, or the complement thereof; a thymine at a position corresponding to position 118 according to SEQ ID NO:31, or the complement thereof; a thymine at a position corresponding to position 131 according to SEQ ID NO:32, or the complement thereof; a thymine at a position corresponding to position 131 according to SEQ ID NO:33, or the complement thereof; a thymine at a position corresponding to position 118 according to SEQ ID NO:34, or the complement thereof; a thymine at a position corresponding to position 201 according to SEQ ID NO:35, or the complement thereof; a thymine at a position corresponding to position 201 according to SEQ ID NO:36, or the complement thereof; a thymine at a position corresponding to position 174 according to SEQ ID NO:37, or the complement thereof; or a thymine at a position corresponding to position 191 according to SEQ ID NO:38, or the complement thereof.

In some embodiments, the biological sample comprises a cell or cell lysate. Such methods can further comprise, for example, obtaining a biological sample from the subject comprising a TREM2 or TYROBP genomic nucleic acid molecule or mRNA molecule, and if mRNA, optionally reverse transcribing the mRNA into cDNA. Such assays can comprise, for example determining the identity of these positions of the particular TREM2 or TYROBP nucleic acid molecule. In some embodiments, the method is an in vitro method.

In some embodiments, the determining step, detecting step, or sequence analysis comprises sequencing at least a portion of the nucleotide sequence of the TREM2 or TYROBP genomic nucleic acid molecule, the TREM2 or TYROBP mRNA molecule, or the TREM2 or TYROBP cDNA molecule in the biological sample, wherein the sequenced portion comprises one or more variations of any of the variants described herein.

In some embodiments, the determining step, detecting step, or sequence analysis comprises sequencing at least a portion of: i) the nucleotide sequence of the TREM2 genomic nucleic acid molecule in the biological sample, wherein the sequenced portion comprises a position corresponding to position 1,630 according to SEQ ID NO:2, or the complement thereof; ii) the nucleotide sequence of the TREM2 mRNA molecule in the biological sample, wherein the sequenced portion comprises a position corresponding to: position 201 according to SEQ ID NO:12, or the complement thereof; position 118 according to SEQ ID NO:13, or the complement thereof; position 131 according to SEQ ID NO:14, or the complement thereof; position 131 according to SEQ ID NO:15, or the complement thereof; position 118 according to SEQ ID NO:16, or the complement thereof; position 201 according to SEQ ID NO:17, or the complement thereof; position 201 according to SEQ ID NO:18, or the complement thereof; position 174 according to SEQ ID NO:19, or the complement thereof; or position 191 according to SEQ ID NO:20, or the complement thereof; and/or iii) the nucleotide sequence of the TREM2 cDNA molecule produced from the mRNA in the biological sample, wherein the sequenced portion comprises a position corresponding to: position 201 according to SEQ ID NO:30, or the complement thereof; position 118 according to SEQ ID NO:31, or the complement thereof; position 131 according to SEQ ID NO:32, or the complement thereof; position 131 according to SEQ ID NO:33, or the complement thereof; position 118 according to SEQ ID NO:34, or the complement thereof; position 201 according to SEQ ID NO:35, or the complement thereof; position 201 according to SEQ ID NO:36, or the complement thereof; position 174 according to SEQ ID NO:37, or the complement thereof; or position 191 according to SEQ ID NO:38, or the complement thereof. When the sequenced portion of the TREM2 nucleic acid molecule in the biological sample comprises: i) a thymine at a position corresponding to position 1,630 according to SEQ ID NO:2; ii) a uracil at a position corresponding to position 201 according to SEQ ID NO:12, a uracil at a position corresponding to position 118 according to SEQ ID NO:13, a uracil at a position corresponding to position 131 according to SEQ ID NO:14, a uracil at a position corresponding to position 131 according to SEQ ID NO:15, a uracil at a position corresponding to position 118 according to SEQ ID NO:16, a uracil at a position corresponding to position 201 according to SEQ ID NO:17, a uracil at a position corresponding to position 201 according to SEQ ID NO:18, a uracil at a position corresponding to position 174 according to SEQ ID NO:19, or a uracil at a position corresponding to position 191 according to SEQ ID NO:20; or iii) a thymine at a position corresponding to position 201 according to SEQ ID NO:30, a thymine at a position corresponding to position 118 according to SEQ ID NO:31, a thymine at a position corresponding to position 131 according to SEQ ID NO:32, a thymine at a position corresponding to position 131 according to SEQ ID NO:33, a thymine at a position corresponding to position 118 according to SEQ ID NO:34, a thymine at a position corresponding to position 201 according to SEQ ID NO:35, a thymine at a position corresponding to position 201 according to SEQ ID NO:36, a thymine at a position corresponding to position 174 according to SEQ ID NO:37, or a thymine at a position corresponding to position 191 according to SEQ ID NO:38, then the TREM2 nucleic acid molecule in the biological sample is a TREM2 variant nucleic acid molecule.

In some embodiments, the determining step, detecting step, or sequence analysis comprises sequencing at least a portion of the nucleotide sequence of the TREM2 genomic nucleic acid molecule in the biological sample, wherein the sequenced portion comprises a position corresponding to position 1,630 according to SEQ ID NO:2, or the complement thereof. When the sequenced portion of the TREM2 nucleic acid molecule in the biological sample comprises: i) a thymine at a position corresponding to position 1,630 according to SEQ ID NO:2, then the TREM2 nucleic acid molecule in the biological sample is a TREM2 variant nucleic acid molecule.

In some embodiments, the determining step, detecting step, or sequence analysis comprises sequencing at least a portion of the nucleotide sequence of the TREM2 mRNA molecule in the biological sample, wherein the sequenced portion comprises a position corresponding to: position 201 according to SEQ ID NO:12, or the complement thereof; position 118 according to SEQ ID NO:13, or the complement thereof; position 131 according to SEQ ID NO:14, or the complement thereof; position 131 according to SEQ ID NO:15, or the complement thereof; position 118 according to SEQ ID NO:16, or the complement thereof; position 201 according to SEQ ID NO:17, or the complement thereof; position 201 according to SEQ ID NO:18, or the complement thereof; position 174 according to SEQ ID NO:19, or the complement thereof; or position 191 according to SEQ ID NO:20, or the complement thereof. When the sequenced portion of the TREM2 nucleic acid molecule in the biological sample comprises: a uracil at a position corresponding to position 201 according to SEQ ID NO:12; a uracil at a position corresponding to position 118 according to SEQ ID NO:13; a uracil at a position corresponding to position 131 according to SEQ ID NO:14; a uracil at a position corresponding to position 131 according to SEQ ID NO:15; a uracil at a position corresponding to position 118 according to SEQ ID NO:16; a uracil at a position corresponding to position 201 according to SEQ ID NO:17; a uracil at a position corresponding to position 201 according to SEQ ID NO:18; a uracil at a position corresponding to position 174 according to SEQ ID NO:19; or a uracil at a position corresponding to position 191 according to SEQ ID NO:20; then the TREM2 nucleic acid molecule in the biological sample is a TREM2 variant nucleic acid molecule.

In some embodiments, the determining step, detecting step, or sequence analysis comprises sequencing at least a portion of the nucleotide sequence of the TREM2 cDNA molecule produced from the mRNA molecule in the biological sample, wherein the sequenced portion comprises a position corresponding to: position 201 according to SEQ ID NO:30, or the complement thereof; position 118 according to SEQ ID NO:31, or the complement thereof; position 131 according to SEQ ID NO:32, or the complement thereof; position 131 according to SEQ ID NO:33, or the complement thereof; position 118 according to SEQ ID NO:34, or the complement thereof; position 201 according to SEQ ID NO:35, or the complement thereof; position 201 according to SEQ ID NO:36, or the complement thereof; position 174 according to SEQ ID NO:37, or the complement thereof; or position 191 according to SEQ ID NO:38, or the complement thereof. When the sequenced portion of the TREM2 nucleic acid molecule in the biological sample comprises: a thymine at a position corresponding to position 201 according to SEQ ID NO:30; a thymine at a position corresponding to position 118 according to SEQ ID NO:31; a thymine at a position corresponding to position 131 according to SEQ ID NO:32; a thymine at a position corresponding to position 131 according to SEQ ID NO:33; a thymine at a position corresponding to position 118 according to SEQ ID NO:34; a thymine at a position corresponding to position 201 according to SEQ ID NO:35; a thymine at a position corresponding to position 201 according to SEQ ID NO:36; a thymine at a position corresponding to position 174 according to SEQ ID NO:37; or a thymine at a position corresponding to position 191 according to SEQ ID NO:38; then the TREM2 nucleic acid molecule in the biological sample is a TREM2 variant nucleic acid molecule.

In some embodiments, the determining step, detecting step, or sequence analysis comprises: a) contacting the biological sample with a primer hybridizing to a portion of the nucleotide sequence of the TREM2: i) genomic nucleic acid molecule that is proximate to a position corresponding to position 1,630 according to SEQ ID NO:2; ii) mRNA molecule that is proximate to a position corresponding to: position 201 according to SEQ ID NO:12, position 118 according to SEQ ID NO:13, position 131 according to SEQ ID NO:14, position 131 according to SEQ ID NO:15, position 118 according to SEQ ID NO:16, position 201 according to SEQ ID NO:17, position 201 according to SEQ ID NO:18, position 174 according to SEQ ID NO:19, or position 191 according to SEQ ID NO:20; and/or iii) cDNA molecule that is proximate to a position corresponding to: position 201 according to SEQ ID NO:30, position 118 according to SEQ ID NO:31, position 131 according to SEQ ID NO:32, position 131 according to SEQ ID NO:33, position 118 according to SEQ ID NO:34, position 201 according to SEQ ID NO:35, position 201 according to SEQ ID NO:36, position 174 according to SEQ ID NO:37, or position 191 according to SEQ ID NO:38; b) extending the primer at least through the position of the nucleotide sequence of the TREM2: i) genomic nucleic acid molecule corresponding to position 1,630 according to SEQ ID NO:2; ii) mRNA molecule corresponding to: position 201 according to SEQ ID NO:12, position 118 according to SEQ ID NO:13, position 131 according to SEQ ID NO:14, position 131 according to SEQ ID NO:15, position 118 according to SEQ ID NO:16, position 201 according to SEQ ID NO:17, position 201 according to SEQ ID NO:18, position 174 according to SEQ ID NO:19, or position 191 according to SEQ ID NO:20; and/or iii) cDNA molecule corresponding to: position 201 according to SEQ ID NO:30, position 118 according to SEQ ID NO:31, position 131 according to SEQ ID NO:32, position 131 according to SEQ ID NO:33, position 118 according to SEQ ID NO:34, position 201 according to SEQ ID NO:35, position 201 according to SEQ ID NO:36, position 174 according to SEQ ID NO:37, or position 191 according to SEQ ID NO:38; and c) determining whether the extension product of the primer comprises: i) a thymine at a position corresponding to position 1,630 according to SEQ ID NO:2; ii) a uracil at a position corresponding to position 201 according to SEQ ID NO:12, a uracil at a position corresponding to position 118 according to SEQ ID NO:13, a uracil at a position corresponding to position 131 according to SEQ ID NO:14, a uracil at a position corresponding to position 131 according to SEQ ID NO:15, a uracil at a position corresponding to position 118 according to SEQ ID NO:16, a uracil at a position corresponding to position 201 according to SEQ ID NO:17, a uracil at a position corresponding to position 201 according to SEQ ID NO:18, a uracil at a position corresponding to position 174 according to SEQ ID NO:19, or a uracil at a position corresponding to position 191 according to SEQ ID NO:20; or iii) a thymine at a position corresponding to position 201 according to SEQ ID NO:30, a thymine at a position corresponding to position 118 according to SEQ ID NO:31, a thymine at a position corresponding to position 131 according to SEQ ID NO:32, a thymine at a position corresponding to position 131 according to SEQ ID NO:33, a thymine at a position corresponding to position 118 according to SEQ ID NO:34, a thymine at a position corresponding to position 201 according to SEQ ID NO:35, a thymine at a position corresponding to position 201 according to SEQ ID NO:36, a thymine at a position corresponding to position 174 according to SEQ ID NO:37, or a thymine at a position corresponding to position 191 according to SEQ ID NO:38.

In some embodiments, the determining step, detecting step, or sequence analysis comprises: a) contacting the biological sample with a primer hybridizing to a portion of the nucleotide sequence of the TREM2 genomic nucleic acid molecule that is proximate to a position corresponding to position 1,630 according to SEQ ID NO:2; b) extending the primer at least through the position of the nucleotide sequence of the TREM2 genomic nucleic acid molecule corresponding to position 1,630 according to SEQ ID NO:2; and c) determining whether the extension product of the primer comprises a thymine at a position corresponding to position 1,630 according to SEQ ID NO:2.

In some embodiments, the determining step, detecting step, or sequence analysis comprises: a) contacting the biological sample with a primer hybridizing to a portion of the nucleotide sequence of the TREM2 mRNA molecule that is proximate to a position corresponding to: position 201 according to SEQ ID NO:12, position 118 according to SEQ ID NO:13, position 131 according to SEQ ID NO:14, position 131 according to SEQ ID NO:15, position 118 according to SEQ ID NO:16, position 201 according to SEQ ID NO:17, position 201 according to SEQ ID NO:18, position 174 according to SEQ ID NO:19, or position 191 according to SEQ ID NO:20; b) extending the primer at least through the position of the nucleotide sequence of the TREM2 mRNA molecule corresponding to: position 201 according to SEQ ID NO:12, position 118 according to SEQ ID NO:13, position 131 according to SEQ ID NO:14, position 131 according to SEQ ID NO:15, position 118 according to SEQ ID NO:16, position 201 according to SEQ ID NO:17, position 201 according to SEQ ID NO:18, position 174 according to SEQ ID NO:19, or position 191 according to SEQ ID NO:20; and c) determining whether the extension product of the primer comprises: a uracil at a position corresponding to position 201 according to SEQ ID NO:12, a uracil at a position corresponding to position 118 according to SEQ ID NO:13, a uracil at a position corresponding to position 131 according to SEQ ID NO:14, a uracil at a position corresponding to position 131 according to SEQ ID NO:15, a uracil at a position corresponding to position 118 according to SEQ ID NO:16, a uracil at a position corresponding to position 201 according to SEQ ID NO:17, a uracil at a position corresponding to position 201 according to SEQ ID NO:18, a uracil at a position corresponding to position 174 according to SEQ ID NO:19, or a uracil at a position corresponding to position 191 according to SEQ ID NO:20.

In some embodiments, the determining step, detecting step, or sequence analysis comprises: a) contacting the biological sample with a primer hybridizing to a portion of the nucleotide sequence of the TREM2 CDNA molecule that is proximate to a position corresponding to: position 201 according to SEQ ID NO:30, position 118 according to SEQ ID NO:31, position 131 according to SEQ ID NO:32, position 131 according to SEQ ID NO:33, position 118 according to SEQ ID NO:34, position 201 according to SEQ ID NO:35, position 201 according to SEQ ID NO:36, position 174 according to SEQ ID NO:37, or position 191 according to SEQ ID NO:38; b) extending the primer at least through the position of the nucleotide sequence of the TREM2 CDNA molecule corresponding to: position 201 according to SEQ ID NO:30, position 118 according to SEQ ID NO:31, position 131 according to SEQ ID NO:32, position 131 according to SEQ ID NO:33, position 118 according to SEQ ID NO:34, position 201 according to SEQ ID NO:35, position 201 according to SEQ ID NO:36, position 174 according to SEQ ID NO:37, or position 191 according to SEQ ID NO:38; and c) determining whether the extension product of the primer comprises: a thymine at a position corresponding to position 201 according to SEQ ID NO:30, a thymine at a position corresponding to position 118 according to SEQ ID NO:31, a thymine at a position corresponding to position 131 according to SEQ ID NO:32, a thymine at a position corresponding to position 131 according to SEQ ID NO:33, a thymine at a position corresponding to position 118 according to SEQ ID NO:34, a thymine at a position corresponding to position 201 according to SEQ ID NO:35, a thymine at a position corresponding to position 201 according to SEQ ID NO:36, a thymine at a position corresponding to position 174 according to SEQ ID NO:37, or a thymine at a position corresponding to position 191 according to SEQ ID NO:38.

In some embodiments, the assay comprises sequencing the entire nucleic acid molecule. In some embodiments, only a TREM2 or TYROBP genomic nucleic acid molecule is analyzed. In some embodiments, only a TREM2 or TYROBP mRNA is analyzed. In some embodiments, only a TREM2 cDNA or TYROBP obtained from TREM2 mRNA or TYROBP is analyzed.

In some embodiments, the determining step, detecting step, or sequence analysis comprises: a) amplifying at least a portion of the TREM2 nucleic acid molecule, wherein the amplified portion comprises: i) a thymine at a position corresponding to position 1,630 according to SEQ ID NO:2, or the complement thereof (for genomic nucleic acid molecules); ii) a uracil at a position corresponding to position 201 according to SEQ ID NO:12, or the complement thereof; a uracil at a position corresponding to position 118 according to SEQ ID NO:13, or the complement thereof; a uracil at a position corresponding to position 131 according to SEQ ID NO:14, or the complement thereof; a uracil at a position corresponding to position 131 according to SEQ ID NO:15, or the complement thereof; a uracil at a position corresponding to position 118 according to SEQ ID NO:16, or the complement thereof; a uracil at a position corresponding to position 201 according to SEQ ID NO:17, or the complement thereof; a uracil at a position corresponding to position 201 according to SEQ ID NO:18, or the complement thereof; a uracil at a position corresponding to position 174 according to SEQ ID NO:19; or a uracil at a position corresponding to position 191 according to SEQ ID NO:20, or the complement thereof (for mRNA molecules); and/or iii) a thymine at a position corresponding to position 201 according to SEQ ID NO:30, or the complement thereof; a thymine at a position corresponding to position 118 according to SEQ ID NO:31, or the complement thereof; a thymine at a position corresponding to position 131 according to SEQ ID NO:32, or the complement thereof; a thymine at a position corresponding to position 131 according to SEQ ID NO:33, or the complement thereof; a thymine at a position corresponding to position 118 according to SEQ ID NO:34, or the complement thereof; a thymine at a position corresponding to position 201 according to SEQ ID NO:35, or the complement thereof; a thymine at a position corresponding to position 201 according to SEQ ID NO:36, or the complement thereof; a thymine at a position corresponding to position 174 according to SEQ ID NO:37, or the complement thereof; or a thymine at a position corresponding to position 191 according to SEQ ID NO:38, or the complement thereof (for cDNA molecules); b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: i) a thymine at a position corresponding to position 1,630 according to SEQ ID NO:2, or the complement thereof (for genomic nucleic acid molecules); ii) a uracil at a position corresponding to position 201 according to SEQ ID NO:12, or the complement thereof; a uracil at a position corresponding to position 118 according to SEQ ID NO:13, or the complement thereof; a uracil at a position corresponding to position 131 according to SEQ ID NO:14, or the complement thereof; a uracil at a position corresponding to position 131 according to SEQ ID NO:15, or the complement thereof; a uracil at a position corresponding to position 118 according to SEQ ID NO:16, or the complement thereof; a uracil at a position corresponding to position 201 according to SEQ ID NO:17, or the complement thereof; a uracil at a position corresponding to position 201 according to SEQ ID NO:18, or the complement thereof; a uracil at a position corresponding to position 174 according to SEQ ID NO:19; or a uracil at a position corresponding to position 191 according to SEQ ID NO:20, or the complement thereof (for mRNA molecules); and/or iii) a thymine at a position corresponding to position 201 according to SEQ ID NO:30, or the complement thereof; a thymine at a position corresponding to position 118 according to SEQ ID NO:31, or the complement thereof; a thymine at a position corresponding to position 131 according to SEQ ID NO:32, or the complement thereof; a thymine at a position corresponding to position 131 according to SEQ ID NO:33, or the complement thereof; a thymine at a position corresponding to position 118 according to SEQ ID NO:34, or the complement thereof; a thymine at a position corresponding to position 201 according to SEQ ID NO:35, or the complement thereof; a thymine at a position corresponding to position 201 according to SEQ ID NO:36, or the complement thereof; a thymine at a position corresponding to position 174 according to SEQ ID NO:37, or the complement thereof; or a thymine at a position corresponding to position 191 according to SEQ ID NO:38, or the complement thereof (for cDNA molecules); and d) detecting the detectable label.

In some embodiments, the determining step, detecting step, or sequence analysis comprises: a) amplifying at least a portion of the TREM2 genomic nucleic acid molecule, wherein the amplified portion comprises a thymine at a position corresponding to position 1,630 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a thymine at a position corresponding to position 1,630 according to SEQ ID NO:2, or the complement thereof; and d) detecting the detectable label.

In some embodiments, the determining step, detecting step, or sequence analysis comprises: a) amplifying at least a portion of the TREM2 mRNA molecule, wherein the amplified portion comprises: a uracil at a position corresponding to position 201 according to SEQ ID NO:12, or the complement thereof; a uracil at a position corresponding to position 118 according to SEQ ID NO:13, or the complement thereof; a uracil at a position corresponding to position 131 according to SEQ ID NO:14, or the complement thereof; a uracil at a position corresponding to position 131 according to SEQ ID NO:15, or the complement thereof; a uracil at a position corresponding to position 118 according to SEQ ID NO:16, or the complement thereof; a uracil at a position corresponding to position 201 according to SEQ ID NO:17, or the complement thereof; a uracil at a position corresponding to position 201 according to SEQ ID NO:18, or the complement thereof; a uracil at a position corresponding to position 174 according to SEQ ID NO:19, or the complement thereof; or a uracil at a position corresponding to position 191 according to SEQ ID NO:20 or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a uracil at a position corresponding to position 201 according to SEQ ID NO:12, or the complement thereof; a uracil at a position corresponding to position 118 according to SEQ ID NO:13, or the complement thereof; a uracil at a position corresponding to position 131 according to SEQ ID NO:14, or the complement thereof; a uracil at a position corresponding to position 131 according to SEQ ID NO:15, or the complement thereof; a uracil at a position corresponding to position 118 according to SEQ ID NO:16, or the complement thereof; a uracil at a position corresponding to position 201 according to SEQ ID NO:17, or the complement thereof; a uracil at a position corresponding to position 201 according to SEQ ID NO:18, or the complement thereof; a uracil at a position corresponding to position 174 according to SEQ ID NO:19, or the complement thereof; or a uracil at a position corresponding to position 191 according to SEQ ID NO:20 or the complement thereof; and d) detecting the detectable label.

In some embodiments, the determining step, detecting step, or sequence analysis comprises: a) amplifying at least a portion of the TREM2 cDNA molecule, wherein the amplified portion comprises: a thymine at a position corresponding to position 201 according to SEQ ID NO:30, or the complement thereof; a thymine at a position corresponding to position 118 according to SEQ ID NO:31, or the complement thereof; a thymine at a position corresponding to position 131 according to SEQ ID NO:32, or the complement thereof; a thymine at a position corresponding to position 131 according to SEQ ID NO:33, or the complement thereof; a thymine at a position corresponding to position 118 according to SEQ ID NO:34, or the complement thereof; a thymine at a position corresponding to position 201 according to SEQ ID NO:35, or the complement thereof; a thymine at a position corresponding to position 201 according to SEQ ID NO:36, or the complement thereof; a thymine at a position corresponding to position 174 according to SEQ ID NO:37, or the complement thereof; or a thymine at a position corresponding to position 191 according to SEQ ID NO:38, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a thymine at a position corresponding to position 201 according to SEQ ID NO:30, or the complement thereof; a thymine at a position corresponding to position 118 according to SEQ ID NO:31, or the complement thereof; a thymine at a position corresponding to position 131 according to SEQ ID NO:32, or the complement thereof; a thymine at a position corresponding to position 131 according to SEQ ID NO:33, or the complement thereof; a thymine at a position corresponding to position 118 according to SEQ ID NO:34, or the complement thereof; a thymine at a position corresponding to position 201 according to SEQ ID NO:35, or the complement thereof; a thymine at a position corresponding to position 201 according to SEQ ID NO:36, or the complement thereof; a thymine at a position corresponding to position 174 according to SEQ ID NO:37, or the complement thereof; or a thymine at a position corresponding to position 191 according to SEQ ID NO:38, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: i) a thymine at a position corresponding to position 1,630 according to SEQ ID NO:2, or the complement thereof (for genomic nucleic acid molecules); ii) a uracil at a position corresponding to position 201 according to SEQ ID NO:12, or the complement thereof; a uracil at a position corresponding to position 118 according to SEQ ID NO:13, or the complement thereof; a uracil at a position corresponding to position 131 according to SEQ ID NO:14, or the complement thereof; a uracil at a position corresponding to position 131 according to SEQ ID NO:15, or the complement thereof; a uracil at a position corresponding to position 118 according to SEQ ID NO:16, or the complement thereof; a uracil at a position corresponding to position 201 according to SEQ ID NO:17, or the complement thereof; a uracil at a position corresponding to position 201 according to SEQ ID NO:18, or the complement thereof; a uracil at a position corresponding to position 174 according to SEQ ID NO:19; or a uracil at a position corresponding to position 191 according to SEQ ID NO:20, or the complement thereof (for mRNA molecules); and/or iii) a thymine at a position corresponding to position 201 according to SEQ ID NO:30, or the complement thereof; a thymine at a position corresponding to position 118 according to SEQ ID NO:31, or the complement thereof; a thymine at a position corresponding to position 131 according to SEQ ID NO:32, or the complement thereof; a thymine at a position corresponding to position 131 according to SEQ ID NO:33, or the complement thereof; a thymine at a position corresponding to position 118 according to SEQ ID NO:34, or the complement thereof; a thymine at a position corresponding to position 201 according to SEQ ID NO:35, or the complement thereof; a thymine at a position corresponding to position 201 according to SEQ ID NO:36, or the complement thereof; a thymine at a position corresponding to position 174 according to SEQ ID NO:37, or the complement thereof; or a thymine at a position corresponding to position 191 according to SEQ ID NO:38, or the complement thereof (for cDNA molecules); and detecting the detectable label.

In some embodiments, the determining step, detecting step, or sequence analysis comprises: contacting the genomic nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a thymine at a position corresponding to position 1,630 according to SEQ ID NO:2, or the complement thereof; and detecting the detectable label.

In some embodiments, the determining step, detecting step, or sequence analysis comprises: contacting the mRNA molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a uracil at a position corresponding to position 201 according to SEQ ID NO:12, or the complement thereof; a uracil at a position corresponding to position 118 according to SEQ ID NO:13, or the complement thereof; a uracil at a position corresponding to position 131 according to SEQ ID NO:14, or the complement thereof; a uracil at a position corresponding to position 131 according to SEQ ID NO:15, or the complement thereof; a uracil at a position corresponding to position 118 according to SEQ ID NO:16, or the complement thereof; a uracil at a position corresponding to position 201 according to SEQ ID NO:17, or the complement thereof; a uracil at a position corresponding to position 201 according to SEQ ID NO:18, or the complement thereof; a uracil at a position corresponding to position 174 according to SEQ ID NO:19, or the complement thereof; or a uracil at a position corresponding to position 191 according to SEQ ID NO:20 or the complement thereof; and detecting the detectable label.

In some embodiments, the determining step, detecting step, or sequence analysis comprises: contacting the cDNA molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a thymine at a position corresponding to position 201 according to SEQ ID NO:30, or the complement thereof; a thymine at a position corresponding to position 118 according to SEQ ID NO:31, or the complement thereof; a thymine at a position corresponding to position 131 according to SEQ ID NO:32, or the complement thereof; a thymine at a position corresponding to position 131 according to SEQ ID NO:33, or the complement thereof; a thymine at a position corresponding to position 118 according to SEQ ID NO:34, or the complement thereof; a thymine at a position corresponding to position 201 according to SEQ ID NO:35, or the complement thereof; a thymine at a position corresponding to position 201 according to SEQ ID NO:36, or the complement thereof; a thymine at a position corresponding to position 174 according to SEQ ID NO:37, or the complement thereof; or a thymine at a position corresponding to position 191 according to SEQ ID NO:38, or the complement thereof; and detecting the detectable label.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to a TREM2 or TYROBP variant genomic sequence, variant mRNA sequence, or variant cDNA sequence and not the corresponding TREM2 or TYROBP reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a TREM2 or TYROBP variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether a TREM2 nucleic acid molecule (genomic nucleic acid molecule, mRNA molecule, or cDNA molecule), or complement thereof, within a biological sample comprises a nucleotide sequence comprising a thymine at a position corresponding to position 1,630 according to SEQ ID NO:2 (genomic nucleic acid molecule), a uracil at a position corresponding to position 201 according to SEQ ID NO:12; a uracil at a position corresponding to position 118 according to SEQ ID NO:13; a uracil at a position corresponding to position 131 according to SEQ ID NO:14; a uracil at a position corresponding to position 131 according to SEQ ID NO:15; a uracil at a position corresponding to position 118 according to SEQ ID NO:16; a uracil at a position corresponding to position 201 according to SEQ ID NO:17; a uracil at a position corresponding to position 201 according to SEQ ID NO:18; a uracil at a position corresponding to position 174 according to SEQ ID NO:19; or a uracil at a position corresponding to position 191 according to SEQ ID NO:20 (for mRNA molecules); a thymine at a position corresponding to position 201 according to SEQ ID NO:30; a thymine at a position corresponding to position 118 according to SEQ ID NO:31; a thymine at a position corresponding to position 131 according to SEQ ID NO:32; a thymine at a position corresponding to position 131 according to SEQ ID NO:33; a thymine at a position corresponding to position 118 according to SEQ ID NO:34; a thymine at a position corresponding to position 201 according to SEQ ID NO:35; a thymine at a position corresponding to position 201 according to SEQ ID NO:36; a thymine at a position corresponding to position 174 according to SEQ ID NO:37; or a thymine at a position corresponding to position 191 according to SEQ ID NO:38 (for CDNA molecules), the biological sample can be subjected to an amplification method using a primer pair that includes a first primer derived from the 5′ flanking sequence adjacent to a thymine at a position corresponding to position 1,630 according to SEQ ID NO:2, a uracil at a position corresponding to position 201 according to SEQ ID NO:12, a uracil at a position corresponding to position 118 according to SEQ ID NO:13, a uracil at a position corresponding to position 131 according to SEQ ID NO:14, a uracil at a position corresponding to position 131 according to SEQ ID NO:15, a uracil at a position corresponding to position 118 according to SEQ ID NO:16, a uracil at a position corresponding to position 201 according to SEQ ID NO:17, a uracil at a position corresponding to position 201 according to SEQ ID NO:18, a uracil at a position corresponding to position 174 according to SEQ ID NO:19, or a uracil at a position corresponding to position 191 according to SEQ ID NO:20, a thymine at a position corresponding to position 201 according to SEQ ID NO:30, a thymine at a position corresponding to position 118 according to SEQ ID NO:31, a thymine at a position corresponding to position 131 according to SEQ ID NO:32, a thymine at a position corresponding to position 131 according to SEQ ID NO:33, a thymine at a position corresponding to position 118 according to SEQ ID NO:34, a thymine at a position corresponding to position 201 according to SEQ ID NO:35, a thymine at a position corresponding to position 201 according to SEQ ID NO:36, a thymine at a position corresponding to position 174 according to SEQ ID NO:37, or a thymine at a position corresponding to position 191 according to SEQ ID NO:38, and a second primer derived from the 3′ flanking sequence adjacent to a thymine at a position corresponding to position 1,630 according to SEQ ID NO:2, a uracil at a position corresponding to position 201 according to SEQ ID NO:12, a uracil at a position corresponding to position 118 according to SEQ ID NO:13, a uracil at a position corresponding to position 131 according to SEQ ID NO:14, a uracil at a position corresponding to position 131 according to SEQ ID NO:15, a uracil at a position corresponding to position 118 according to SEQ ID NO:16, a uracil at a position corresponding to position 201 according to SEQ ID NO:17, a uracil at a position corresponding to position 201 according to SEQ ID NO:18, a uracil at a position corresponding to position 174 according to SEQ ID NO:19, or a uracil at a position corresponding to position 191 according to SEQ ID NO:20, a thymine at a position corresponding to position 201 according to SEQ ID NO:30, a thymine at a position corresponding to position 118 according to SEQ ID NO:31, a thymine at a position corresponding to position 131 according to SEQ ID NO:32, a thymine at a position corresponding to position 131 according to SEQ ID NO:33, a thymine at a position corresponding to position 118 according to SEQ ID NO:34, a thymine at a position corresponding to position 201 according to SEQ ID NO:35, a thymine at a position corresponding to position 201 according to SEQ ID NO:36, a thymine at a position corresponding to position 174 according to SEQ ID NO:37; or a thymine at a position corresponding to position 191 according to SEQ ID NO:38 to produce an amplicon that is indicative of the presence of the SNP at positions encoding a thymine at a position corresponding to position 1,630 according to SEQ ID NO:2, a uracil at a position corresponding to position 201 according to SEQ ID NO:12, a uracil at a position corresponding to position 118 according to SEQ ID NO:13, a uracil at a position corresponding to position 131 according to SEQ ID NO:14, a uracil at a position corresponding to position 131 according to SEQ ID NO:15, a uracil at a position corresponding to position 118 according to SEQ ID NO:16, a uracil at a position corresponding to position 201 according to SEQ ID NO:17, a uracil at a position corresponding to position 201 according to SEQ ID NO:18, a uracil at a position corresponding to position 174 according to SEQ ID NO:19, or a uracil at a position corresponding to position 191 according to SEQ ID NO:20, a thymine at a position corresponding to position 201 according to SEQ ID NO:30, a thymine at a position corresponding to position 118 according to SEQ ID NO:31, a thymine at a position corresponding to position 131 according to SEQ ID NO:32, a thymine at a position corresponding to position 131 according to SEQ ID NO:33, a thymine at a position corresponding to position 118 according to SEQ ID NO:34, a thymine at a position corresponding to position 201 according to SEQ ID NO:35, a thymine at a position corresponding to position 201 according to SEQ ID NO:36, a thymine at a position corresponding to position 174 according to SEQ ID NO:37, or a thymine at a position corresponding to position 191 according to SEQ ID NO:38. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 1,630 according to SEQ ID NO:2, a uracil at a position corresponding to position 201 according to SEQ ID NO:12, a uracil at a position corresponding to position 118 according to SEQ ID NO:13, a uracil at a position corresponding to position 131 according to SEQ ID NO:14, a uracil at a position corresponding to position 131 according to SEQ ID NO:15, a uracil at a position corresponding to position 118 according to SEQ ID NO:16, a uracil at a position corresponding to position 201 according to SEQ ID NO:17, a uracil at a position corresponding to position 201 according to SEQ ID NO:18, a uracil at a position corresponding to position 174 according to SEQ ID NO:19, or a uracil at a position corresponding to position 191 according to SEQ ID NO:20, a thymine at a position corresponding to position 201 according to SEQ ID NO:30, a thymine at a position corresponding to position 118 according to SEQ ID NO:31, a thymine at a position corresponding to position 131 according to SEQ ID NO:32, a thymine at a position corresponding to position 131 according to SEQ ID NO:33, a thymine at a position corresponding to position 118 according to SEQ ID NO:34, a thymine at a position corresponding to position 201 according to SEQ ID NO:35, a thymine at a position corresponding to position 201 according to SEQ ID NO:36, a thymine at a position corresponding to position 174 according to SEQ ID NO:37, or a thymine at a position corresponding to position 191 according to SEQ ID NO:38, and at least 1, 2, 3, 4, 5, 6, 7, 8, 9, 10, or more nucleotides on each side of positions comprising a thymine at a position corresponding to position 1,630 according to SEQ ID NO:2, a uracil at a position corresponding to position 201 according to SEQ ID NO:12, a uracil at a position corresponding to position 118 according to SEQ ID NO:13, a uracil at a position corresponding to position 131 according to SEQ ID NO:14, a uracil at a position corresponding to position 131 according to SEQ ID NO:15, a uracil at a position corresponding to position 118 according to SEQ ID NO:16, a uracil at a position corresponding to position 201 according to SEQ ID NO:17, a uracil at a position corresponding to position 201 according to SEQ ID NO:18, a uracil at a position corresponding to position 174 according to SEQ ID NO:19, or a uracil at a position corresponding to position 191 according to SEQ ID NO:20, a thymine at a position corresponding to position 201 according to SEQ ID NO:30, a thymine at a position corresponding to position 118 according to SEQ ID NO:31, a thymine at a position corresponding to position 131 according to SEQ ID NO:32, a thymine at a position corresponding to position 131 according to SEQ ID NO:33, a thymine at a position corresponding to position 118 according to SEQ ID NO:34, a thymine at a position corresponding to position 201 according to SEQ ID NO:35, a thymine at a position corresponding to position 201 according to SEQ ID NO:36, a thymine at a position corresponding to position 174 according to SEQ ID NO:37, or a thymine at a position corresponding to position 191 according to SEQ ID NO:38.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a human TREM2 predicted loss-of-function polypeptide comprising performing an assay on a biological sample obtained from a subject to determine whether a TREM2 polypeptide in the subject contains one or more variations that causes the polypeptide to have a loss-of-function (partial or complete) or predicted loss-of-function (partial or complete). The TREM2 predicted loss-of-function polypeptide can be any of the TREM2 variant polypeptides described herein. In some embodiments, the methods detect the presence of TREM2 Gln33STOP or Gln63STOP.

In some embodiments, the methods comprise performing an assay on a sample obtained from a subject to determine whether a TREM2 polypeptide in the sample terminates at a position corresponding to position 32 according to SEQ ID NO:43 or position 62 according to SEQ ID NO:44.

In some embodiments, the detecting step comprises sequencing at least a portion of the polypeptide that comprises a position corresponding to position 33 according to SEQ ID NO:43 or SEQ ID NO:39, or position 63 according to SEQ ID NO:44 or SEQ ID NO:42.

In some embodiments, the detecting step comprises sequencing the entire TREM2 polypeptide. In some embodiments, the sequenced TREM2 polypeptide comprises SEQ ID NO:43 or SEQ ID NO:44. In some embodiments, the sequenced TREM2 polypeptide consists of SEQ ID NO:43 or SEQ ID NO:44. In some embodiments, the detecting step comprises sequencing at least a portion of a TREM2 polypeptide that may comprise positions corresponding to any positions that are C-terminal to: position 32 according to SEQ ID NO:43 or position 62 according to SEQ ID NO:44. If amino acids are detected in the TREM2 polypeptide at positions corresponding to: positions 33-219 according to SEQ ID NO:39, or positions 63-249 according to SEQ ID NO:42, then such TREM2 polypeptide is a TREM2 reference polypeptide. An absence of positions 33-219 according to SEQ ID NO:39 in the TREM2 polypeptide indicates that the TREM2 polypeptide terminates at position 32 according to SEQ ID NO:43 and is a TREM2 predicted loss-of-function polypeptide. An absence of positions 63-249 according to SEQ ID NO:42 in the TREM2 polypeptide indicates that the TREM2 polypeptide terminates at position 62 according to SEQ ID NO:44 and is a TREM2 predicted loss-of-function polypeptide.

In some embodiments, the detecting step comprises sequencing the entire TREM2 polypeptide.

In some embodiments, the detecting step comprises an immunoassay for detecting the presence of a TREM2 polypeptide that comprises or consists of SEQ ID NO:43 or SEQ ID NO:44. In some embodiments, the TREM2 polypeptide comprises SEQ ID NO:43 or SEQ ID NO:44. In some embodiments, the TREM2 polypeptide consists of SEQ ID NO:43 or SEQ ID NO:44. In some embodiments, the detecting step comprises detecting at least a portion of a TREM2 polypeptide that may comprise positions corresponding to any positions that are C-terminal to: position 32 according to SEQ ID NO:43 or position 62 according to SEQ ID NO:44. If amino acids are detected in the TREM2 polypeptide at positions corresponding to: positions 33-219 according to SEQ ID NO:39 or positions 63-249 according to SEQ ID NO:42, then such TREM2 polypeptide is a TREM2 reference polypeptide. A lack of detection of positions 33-219 according to SEQ ID NO:39 in the TREM2 polypeptide indicates that the TREM2 polypeptide terminates at position 32 according to SEQ ID NO:43 and is a TREM2 predicted loss-of-function polypeptide. A lack of detection of positions 63-249 according to SEQ ID NO:42 in the TREM2 polypeptide indicates that the TREM2 polypeptide terminates at position 62 according to SEQ ID NO:44 and is a TREM2 predicted loss-of-function polypeptide.

In any of the embodiments described herein, the TREM2 predicted loss-of-function polypeptide can be any of the TREM2 polypeptides described herein including, for example, TREM2 Gln33STOP or Gln63STOP. In some embodiments, the TREM2 predicted loss-of-function variant polypeptide is TREM2 Gln33STOP. In some embodiments, the TREM2 predicted loss-of-function variant polypeptide is Gln63STOP.

In some embodiments, when the subject does not have a TREM2 predicted loss-of-function polypeptide, the subject does not have an increased risk for developing Alzheimer's Disease. In some embodiments, when the subject has a TREM2 predicted loss-of-function polypeptide, the subject has an increased risk for developing Alzheimer's Disease.

The present disclosure also provides isolated nucleic acid molecules that hybridize to TREM2 or TYROBP variant genomic nucleic acid molecules, TREM2 or TYROBP variant mRNA molecules, and/or TREM2 or TYROBP variant cDNA molecules (such as any of the genomic variant nucleic acid molecules, mRNA variant molecules, and cDNA variant molecules disclosed herein).

In some embodiments, the isolated nucleic acid molecules hybridize to a portion of the TREM2 nucleic acid molecule that includes a position corresponding to: i) position 1,630 according to SEQ ID NO:2 (for genomic nucleic acid molecules); ii) position 201 according to SEQ ID NO:12, position 118 according to SEQ ID NO:13, position 131 according to SEQ ID NO:14, position 131 according to SEQ ID NO:15, position 118 according to SEQ ID NO:16, position 201 according to SEQ ID NO:17, position 201 according to SEQ ID NO:18, position 174 according to SEQ ID NO:19, or position 191 according to SEQ ID NO:20 (for mRNA molecules); or iii) position 201 according to SEQ ID NO:30, position 118 according to SEQ ID NO:31, position 131 according to SEQ ID NO:32, position 131 according to SEQ ID NO:33, position 118 according to SEQ ID NO:34, position 201 according to SEQ ID NO:35, position 201 according to SEQ ID NO:36, position 174 according to SEQ ID NO:37, or position 191 according to SEQ ID NO:38 (for cDNA molecules).

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to TREM2 or TYROBP variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to TREM2 or TYROBP variant genomic nucleic acid molecules, TREM2 or TYROBP variant mRNA molecules, and/or TREM2 or TYROBP variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of a TREM2 nucleotide sequence, wherein the portion comprises a position corresponding to: i) position 1,630 according to SEQ ID NO:2, or the complement thereof (for genomic nucleic acid molecules); ii) position 201 according to SEQ ID NO:12, or the complement thereof; position 118 according to SEQ ID NO:13, or the complement thereof; position 131 according to SEQ ID NO:14, or the complement thereof; position 131 according to SEQ ID NO:15, or the complement thereof; position 118 according to SEQ ID NO:16, or the complement thereof; position 201 according to SEQ ID NO:17, or the complement thereof; position 201 according to SEQ ID NO:18, or the complement thereof; position 174 according to SEQ ID NO:19, or the complement thereof; or position 191 according to SEQ ID NO:20, or the complement thereof (for mRNA molecules); and/or iii) position 201 according to SEQ ID NO:30, or the complement thereof; position 118 according to SEQ ID NO:31, or the complement thereof; position 131 according to SEQ ID NO:32, or the complement thereof; position 131 according to SEQ ID NO:33, or the complement thereof; position 118 according to SEQ ID NO:34, or the complement thereof; position 201 according to SEQ ID NO:35, or the complement thereof; position 201 according to SEQ ID NO:36, or the complement thereof; position 174 according to SEQ ID NO:37, or the complement thereof; or position 191 according to SEQ ID NO:38, or the complement thereof (for cDNA molecules). In some embodiments, the alteration-specific probe or alteration-specific primer comprises a nucleotide sequence which is complementary to a portion of a nucleotide sequence comprising positions corresponding to: i) positions 1,630-1,632 according to SEQ ID NO:2, or the complement thereof (for genomic nucleic acid molecules); ii) positions 201-203 according to SEQ ID NO:12, or the complement thereof; positions 118-120 according to SEQ ID NO:13, or the complement thereof; positions 131-133 according to SEQ ID NO:14, or the complement thereof; positions 131-133 according to SEQ ID NO:15, or the complement thereof; positions 118-120 according to SEQ ID NO:16, or the complement thereof; positions 201-203 according to SEQ ID NO:17, or the complement thereof; positions 201-203 according to SEQ ID NO:18, or the complement thereof; positions 174-176 according to SEQ ID NO:19, or the complement thereof; or positions 191-193 according to SEQ ID NO:20, or the complement thereof (for mRNA molecules); and/or iii) positions 201-203 according to SEQ ID NO:30, or the complement thereof; positions 118-120 according to SEQ ID NO:31, or the complement thereof; positions 131-133 according to SEQ ID NO:32, or the complement thereof; positions 131-133 according to SEQ ID NO:33, or the complement thereof; positions 118-120 according to SEQ ID NO:34, or the complement thereof; positions 201-203 according to SEQ ID NO:35, or the complement thereof; positions 201-203 according to SEQ ID NO:36, or the complement thereof; positions 174-176 according to SEQ ID NO:37, or the complement thereof; or positions 191-193 according to SEQ ID NO:38, or the complement thereof (for cDNA molecules).

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the TREM2 or TYROBP variant genomic nucleic acid molecules, TREM2 or TYROBP variant mRNA molecules, and/or TREM2 or TYROBP variant cDNA molecules disclosed herein. The primers described herein can be used to amplify TREM2 or TYROBP variant genomic nucleic acid molecules, TREM2 or TYROBP variant mRNA molecules, or TREM2 or TYROBP variant cDNA molecules, or a fragment thereof.

The present disclosure also provides pairs of primers comprising any of the primers described above. For example, if one of the primers' 3′-ends hybridizes to a cytosine at a position corresponding to position 1,630 according to SEQ ID NO:1 (rather than thymine) in a particular TREM2 nucleic acid molecule, then the presence of the amplified fragment would indicate the presence of a TREM2 reference genomic nucleic acid molecule. Conversely, if one of the primers' 3′-ends hybridizes to a thymine at a position corresponding to position 1,630 according to SEQ ID NO:2 (rather than cytosine) in a particular TREM2 nucleic acid molecule, then the presence of the amplified fragment would indicate the presence of the TREM2 variant genomic nucleic acid molecule. In some embodiments, the nucleotide of the primer complementary to the thymine at a position corresponding to position 1,630 according to SEQ ID NO:2 can be at the 3′ end of the primer. In addition, if one of the primers' 3′-ends hybridizes to a cytosine at a position corresponding to position 201 according to SEQ ID NO:3 (rather than uracil at position 201) in a particular TREM2 nucleic acid molecule, then the presence of the amplified fragment would indicate the presence of a TREM2 reference mRNA molecule. Conversely, if one of the primers' 3′-ends hybridizes to a uracil at a position corresponding to position 201 according to SEQ ID NO:12 (rather than cytosine) in a particular TREM2 mRNA molecule, then the presence of the amplified fragment would indicate the presence of the TREM2 variant mRNA molecule. In some embodiments, the nucleotide of the primer complementary to the uracil at a position corresponding to position 201 according to SEQ ID NO:12 can be at the 3′ end of the primer. In addition, if one of the primers' 3′-ends hybridizes to a cytosine at a position corresponding to position 118 according to SEQ ID NO:4 (rather than uracil at position 118) in a particular TREM2 nucleic acid molecule, then the presence of the amplified fragment would indicate the presence of a TREM2 reference mRNA molecule. Conversely, if one of the primers' 3′-ends hybridizes to a uracil at a position corresponding to position 118 according to SEQ ID NO:13 (rather than cytosine) in a particular TREM2 mRNA molecule, then the presence of the amplified fragment would indicate the presence of the TREM2 variant mRNA molecule. In some embodiments, the nucleotide of the primer complementary to the uracil at a position corresponding to position 118 according to SEQ ID NO:13 can be at the 3′ end of the primer. In addition, if one of the primers' 3′-ends hybridizes to a cytosine at a position corresponding to position 131 according to SEQ ID NO:5 (rather than uracil at position 131) in a particular TREM2 nucleic acid molecule, then the presence of the amplified fragment would indicate the presence of a TREM2 reference mRNA molecule. Conversely, if one of the primers' 3′-ends hybridizes to a uracil at a position corresponding to position 131 according to SEQ ID NO:14 (rather than cytosine) in a particular TREM2 mRNA molecule, then the presence of the amplified fragment would indicate the presence of the TREM2 variant mRNA molecule. In some embodiments, the nucleotide of the primer complementary to the uracil at a position corresponding to position 131 according to SEQ ID NO:14 can be at the 3′ end of the primer. In addition, if one of the primers' 3′-ends hybridizes to a cytosine at a position corresponding to position 131 according to SEQ ID NO:6 (rather than uracil at position 131) in a particular TREM2 nucleic acid molecule, then the presence of the amplified fragment would indicate the presence of a TREM2 reference mRNA molecule. Conversely, if one of the primers' 3′-ends hybridizes to a uracil at a position corresponding to position 131 according to SEQ ID NO:15 (rather than cytosine) in a particular TREM2 mRNA molecule, then the presence of the amplified fragment would indicate the presence of the TREM2 variant mRNA molecule. In some embodiments, the nucleotide of the primer complementary to the uracil at a position corresponding to position 131 according to SEQ ID NO:15 can be at the 3′ end of the primer. In addition, if one of the primers' 3′-ends hybridizes to a cytosine at a position corresponding to position 118 according to SEQ ID NO:7 (rather than uracil at position 118) in a particular TREM2 nucleic acid molecule, then the presence of the amplified fragment would indicate the presence of a TREM2 reference mRNA molecule. Conversely, if one of the primers' 3′-ends hybridizes to a uracil at a position corresponding to position 118 according to SEQ ID NO:16 (rather than cytosine) in a particular TREM2 mRNA molecule, then the presence of the amplified fragment would indicate the presence of the TREM2 variant mRNA molecule. In some embodiments, the nucleotide of the primer complementary to the uracil at a position corresponding to position 118 according to SEQ ID NO:16 can be at the 3′ end of the primer. In addition, if one of the primers' 3′-ends hybridizes to a cytosine at a position corresponding to position 201 according to SEQ ID NO:8 (rather than uracil at position 201) in a particular TREM2 nucleic acid molecule, then the presence of the amplified fragment would indicate the presence of a TREM2 reference mRNA molecule. Conversely, if one of the primers' 3′-ends hybridizes to a uracil at a position corresponding to position 201 according to SEQ ID NO:17 (rather than cytosine) in a particular TREM2 mRNA molecule, then the presence of the amplified fragment would indicate the presence of the TREM2 variant mRNA molecule. In some embodiments, the nucleotide of the primer complementary to the uracil at a position corresponding to position 201 according to SEQ ID NO:17 can be at the 3′ end of the primer. In addition, if one of the primers' 3′-ends hybridizes to a cytosine at a position corresponding to position 201 according to SEQ ID NO:9 (rather than uracil at position 201) in a particular TREM2 nucleic acid molecule, then the presence of the amplified fragment would indicate the presence of a TREM2 reference mRNA molecule. Conversely, if one of the primers' 3′-ends hybridizes to a uracil at a position corresponding to position 201 according to SEQ ID NO:18 (rather than cytosine) in a particular TREM2 mRNA molecule, then the presence of the amplified fragment would indicate the presence of the TREM2 variant mRNA molecule. In some embodiments, the nucleotide of the primer complementary to the uracil at a position corresponding to position 201 according to SEQ ID NO:18 can be at the 3′ end of the primer. In addition, if one of the primers' 3′-ends hybridizes to a cytosine at a position corresponding to position 174 according to SEQ ID NO:10 (rather than uracil at position 174) in a particular TREM2 nucleic acid molecule, then the presence of the amplified fragment would indicate the presence of a TREM2 reference mRNA molecule. Conversely, if one of the primers' 3′-ends hybridizes to a uracil at a position corresponding to position 174 according to SEQ ID NO:19 (rather than cytosine) in a particular TREM2 mRNA molecule, then the presence of the amplified fragment would indicate the presence of the TREM2 variant mRNA molecule. In some embodiments, the nucleotide of the primer complementary to the uracil at a position corresponding to position 174 according to SEQ ID NO:19 can be at the 3′ end of the primer. In addition, if one of the primers' 3′-ends hybridizes to a cytosine at a position corresponding to position 191 according to SEQ ID NO:10 (rather than uracil at position 191) in a particular TREM2 nucleic acid molecule, then the presence of the amplified fragment would indicate the presence of a TREM2 reference mRNA molecule. Conversely, if one of the primers' 3′-ends hybridizes to a uracil at a position corresponding to position 191 according to SEQ ID NO:20 (rather than cytosine) in a particular TREM2 mRNA molecule, then the presence of the amplified fragment would indicate the presence of the TREM2 variant mRNA molecule. In some embodiments, the nucleotide of the primer complementary to the uracil at a position corresponding to position 191 according to SEQ ID NO:20 can be at the 3′ end of the primer. In addition, if one of the primers' 3′-ends hybridizes to a cytosine at a position corresponding to position 201 according to SEQ ID NO:21 (rather than thymine at position 201) in a particular TREM2 nucleic acid molecule, then the presence of the amplified fragment would indicate the presence of a TREM2 reference cDNA molecule. Conversely, if one of the primers' 3′-ends hybridizes to a thymine at a position corresponding to position 201 according to SEQ ID NO:30 (rather than cytosine) in a particular TREM2 cDNA molecule, then the presence of the amplified fragment would indicate the presence of the TREM2 variant cDNA molecule. In some embodiments, the nucleotide of the primer complementary to the thymine at a position corresponding to position 201 according to SEQ ID NO:30 can be at the 3′ end of the primer. In addition, if one of the primers' 3′-ends hybridizes to a cytosine at a position corresponding to position 118 according to SEQ ID NO:22 (rather than thymine at position 118) in a particular TREM2 nucleic acid molecule, then the presence of the amplified fragment would indicate the presence of a TREM2 reference cDNA molecule. Conversely, if one of the primers' 3′-ends hybridizes to a thymine at a position corresponding to position 118 according to SEQ ID NO:31 (rather than cytosine) in a particular TREM2 cDNA molecule, then the presence of the amplified fragment would indicate the presence of the TREM2 variant cDNA molecule. In some embodiments, the nucleotide of the primer complementary to the thymine at a position corresponding to position 118 according to SEQ ID NO:31 can be at the 3′ end of the primer. In addition, if one of the primers' 3′-ends hybridizes to a cytosine at a position corresponding to position 131 according to SEQ ID NO:23 (rather than thymine at position 131) in a particular TREM2 nucleic acid molecule, then the presence of the amplified fragment would indicate the presence of a TREM2 reference cDNA molecule. Conversely, if one of the primers' 3′-ends hybridizes to a thymine at a position corresponding to position 131 according to SEQ ID NO:32 (rather than cytosine) in a particular TREM2 cDNA molecule, then the presence of the amplified fragment would indicate the presence of the TREM2 variant cDNA molecule. In some embodiments, the nucleotide of the primer complementary to the thymine at a position corresponding to position 131 according to SEQ ID NO:32 can be at the 3′ end of the primer. In addition, if one of the primers' 3′-ends hybridizes to a cytosine at a position corresponding to position 131 according to SEQ ID NO:24 (rather than thymine at position 131) in a particular TREM2 nucleic acid molecule, then the presence of the amplified fragment would indicate the presence of a TREM2 reference cDNA molecule. Conversely, if one of the primers' 3′-ends hybridizes to a thymine at a position corresponding to position 131 according to SEQ ID NO:33 (rather than cytosine) in a particular TREM2 cDNA molecule, then the presence of the amplified fragment would indicate the presence of the TREM2 variant cDNA molecule. In some embodiments, the nucleotide of the primer complementary to the thymine at a position corresponding to position 131 according to SEQ ID NO:33 can be at the 3′ end of the primer. In addition, if one of the primers' 3′-ends hybridizes to a cytosine at a position corresponding to position 118 according to SEQ ID NO:25 (rather than thymine at position 118) in a particular TREM2 nucleic acid molecule, then the presence of the amplified fragment would indicate the presence of a TREM2 reference cDNA molecule. Conversely, if one of the primers' 3′-ends hybridizes to a thymine at a position corresponding to position 118 according to SEQ ID NO:34 (rather than cytosine) in a particular TREM2 cDNA molecule, then the presence of the amplified fragment would indicate the presence of the TREM2 variant cDNA molecule. In some embodiments, the nucleotide of the primer complementary to the thymine at a position corresponding to position 118 according to SEQ ID NO:34 can be at the 3′ end of the primer. In addition, if one of the primers' 3′-ends hybridizes to a cytosine at a position corresponding to position 201 according to SEQ ID NO:26 (rather than thymine at position 201) in a particular TREM2 nucleic acid molecule, then the presence of the amplified fragment would indicate the presence of a TREM2 reference cDNA molecule. Conversely, if one of the primers' 3′-ends hybridizes to a thymine at a position corresponding to position 201 according to SEQ ID NO:35 (rather than cytosine) in a particular TREM2 cDNA molecule, then the presence of the amplified fragment would indicate the presence of the TREM2 variant cDNA molecule. In some embodiments, the nucleotide of the primer complementary to the thymine at a position corresponding to position 201 according to SEQ ID NO:35 can be at the 3′ end of the primer. In addition, if one of the primers' 3′-ends hybridizes to a cytosine at a position corresponding to position 201 according to SEQ ID NO:27 (rather than thymine at position 201) in a particular TREM2 nucleic acid molecule, then the presence of the amplified fragment would indicate the presence of a TREM2 reference cDNA molecule. Conversely, if one of the primers' 3′-ends hybridizes to a thymine at a position corresponding to position 201 according to SEQ ID NO:36 (rather than cytosine) in a particular TREM2 cDNA molecule, then the presence of the amplified fragment would indicate the presence of the TREM2 variant cDNA molecule. In some embodiments, the nucleotide of the primer complementary to the thymine at a position corresponding to position 201 according to SEQ ID NO:36 can be at the 3′ end of the primer. In addition, if one of the primers' 3′-ends hybridizes to a cytosine at a position corresponding to position 174 according to SEQ ID NO:28 (rather than thymine at position 174) in a particular TREM2 nucleic acid molecule, then the presence of the amplified fragment would indicate the presence of a TREM2 reference cDNA molecule. Conversely, if one of the primers' 3′-ends hybridizes to a thymine at a position corresponding to position 174 according to SEQ ID NO:37 (rather than cytosine) in a particular TREM2 cDNA molecule, then the presence of the amplified fragment would indicate the presence of the TREM2 variant cDNA molecule. In some embodiments, the nucleotide of the primer complementary to the thymine at a position corresponding to position 174 according to SEQ ID NO:37 can be at the 3′ end of the primer. In addition, if one of the primers' 3′-ends hybridizes to a cytosine at a position corresponding to position 191 according to SEQ ID NO:28 (rather than thymine at position 191) in a particular TREM2 nucleic acid molecule, then the presence of the amplified fragment would indicate the presence of a TREM2 reference cDNA molecule. Conversely, if one of the primers' 3′-ends hybridizes to a thymine at a position corresponding to position 191 according to SEQ ID NO:38 (rather than cytosine) in a particular TREM2 cDNA molecule, then the presence of the amplified fragment would indicate the presence of the TREM2 variant cDNA molecule. In some embodiments, the nucleotide of the primer complementary to the thymine at a position corresponding to position 191 according to SEQ ID NO:38 can be at the 3′ end of the primer.

In the context of the disclosure “specifically hybridizes” means that the probe or primer (such as, for example, the alteration-specific probe or alteration-specific primer) does not hybridize to a nucleic acid sequence encoding a TREM2 or TYROBP reference genomic nucleic acid molecule, a TREM2 or TYROBP reference mRNA molecule, and/or a TREM2 or TYROBP reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleotide sequence of a TREM2 reference genomic nucleic acid molecule is set forth in SEQ ID NO:1. Referring to SEQ ID NO:1, position 1,630 is a cytosine, and codon positions 1,630-1,632 is a CAG codon.

A variant genomic nucleic acid molecule of TREM2 exists, wherein the cytosine at position 1,630 according to SEQ ID NO:1 is replaced with thymine, and the CAG codon at positions 1,630-1,632 according to SEQ ID NO:1 is replaced with TAG. The nucleotide sequence of this TREM2 variant genomic nucleic acid molecule is set forth in SEQ ID NO:2.

The nucleotide sequence of a TYROBP reference genomic nucleic acid molecule is set forth in SEQ ID NO:45.

The nucleotide sequence of a TREM2 reference mRNA molecule (Isoform 1) is set forth in SEQ ID NO:3. Referring to SEQ ID NO:3, position 201 is a cytosine, and codon positions 201-203 is a CAG codon.

The nucleotide sequence of a TREM2 reference mRNA molecule (Isoform 2) is set forth in SEQ ID NO:4. Referring to SEQ ID NO:4, position 118 is a cytosine, and codon positions 118-120 is a CAG codon.

The nucleotide sequence of a TREM2 reference mRNA molecule (Isoform 3) is set forth in SEQ ID NO:5. Referring to SEQ ID NO:5, position 131 is a cytosine, and codon positions 131-133 is a CAG codon.

The nucleotide sequence of a TREM2 reference mRNA molecule (Isoform 4) is set forth in SEQ ID NO:6. Referring to SEQ ID NO:6, position 131 is a cytosine, and codon positions 131-133 is a CAG codon.

The nucleotide sequence of a TREM2 reference mRNA molecule (Isoform 5) is set forth in SEQ ID NO:7. Referring to SEQ ID NO:7, position 118 is a cytosine, and codon positions 118-120 is a CAG codon.

The nucleotide sequence of a TREM2 reference mRNA molecule (Isoform 6) is set forth in SEQ ID NO:8. Referring to SEQ ID NO:8, position 201 is a cytosine, and codon positions 131-133 is a CAG codon.

The nucleotide sequence of a TREM2 reference mRNA molecule (Isoform 7) is set forth in SEQ ID NO:9. Referring to SEQ ID NO:9, position 201 is a cytosine, and codon positions 131-133 is a CAG codon.

The nucleotide sequence of a TREM2 reference mRNA molecule (Isoform 8) is set forth in SEQ ID NO:10. Referring to SEQ ID NO:10, position 174 is a cytosine, and codon positions 174-176 is a CAG codon.

The nucleotide sequence of a TREM2 reference mRNA molecule (Isoform 9) is set forth in SEQ ID NO:11. Referring to SEQ ID NO:11, position 191 is a cytosine, and codon positions 191-193 is a CAG codon.

A variant mRNA molecule of TREM2 exists, wherein the cytosine at position 201 according to SEQ ID NO:3 is replaced with uracil, and the CAG codon at positions 201-203 according to SEQ ID NO:3 is replaced with UAG. The nucleotide sequence of this TREM2 variant mRNA molecule is set forth in SEQ ID NO:12.

Another variant mRNA molecule of TREM2 exists, wherein the cytosine at position 118 according to SEQ ID NO:4 is replaced with uracil, and the CAG codon at positions 118-120 according to SEQ ID NO:4 is replaced with UAG. The nucleotide sequence of this TREM2 variant mRNA molecule is set forth in SEQ ID NO:13.

Another variant mRNA molecule of TREM2 exists, wherein the cytosine at position 131 according to SEQ ID NO:5 is replaced with uracil, and the CAG codon at positions 131-133 according to SEQ ID NO:5 is replaced with UAG. The nucleotide sequence of this TREM2 variant mRNA molecule is set forth in SEQ ID NO:14.

Another variant mRNA molecule of TREM2 exists, wherein the cytosine at position 131 according to SEQ ID NO:6 is replaced with uracil, and the CAG codon at positions 131-133 according to SEQ ID NO:6 is replaced with UAG. The nucleotide sequence of this TREM2 variant mRNA molecule is set forth in SEQ ID NO:15.

A variant mRNA molecule of TREM2 exists, wherein the cytosine at position 118 according to SEQ ID NO:7 is replaced with uracil, and the CAG codon at positions 118-120 according to SEQ ID NO:7 is replaced with UAG. The nucleotide sequence of this TREM2 variant mRNA molecule is set forth in SEQ ID NO:16.

Another variant mRNA molecule of TREM2 exists, wherein the cytosine at position 201 according to SEQ ID NO:8 is replaced with uracil, and the CAG codon at positions 131-133 according to SEQ ID NO:8 is replaced with UAG. The nucleotide sequence of this TREM2 variant mRNA molecule is set forth in SEQ ID NO:17.

Another variant mRNA molecule of TREM2 exists, wherein the cytosine at position 201 according to SEQ ID NO:9 is replaced with uracil, and the CAG codon at positions 131-133 according to SEQ ID NO:9 is replaced with UAG. The nucleotide sequence of this TREM2 variant mRNA molecule is set forth in SEQ ID NO:18.

Another variant mRNA molecule of TREM2 exists, wherein the cytosine at position 174 according to SEQ ID NO:10 is replaced with uracil, and the CAG codon at positions 174-176 according to SEQ ID NO:10 is replaced with UAG. The nucleotide sequence of this TREM2 variant mRNA molecule is set forth in SEQ ID NO:19.

Another variant mRNA molecule of TREM2 exists, wherein the cytosine at position 191 according to SEQ ID NO:11 is replaced with uracil, and the CAG codon at positions 191-193 according to SEQ ID NO:11 is replaced with UAG. The nucleotide sequence of this TREM2 variant mRNA molecule is set forth in SEQ ID NO:20.

The nucleotide sequence of a TYROBP reference mRNA molecule (Isoform 1) is set forth in SEQ ID NO:46. The nucleotide sequence of a TYROBP reference mRNA molecule (Isoform 2) is set forth in SEQ ID NO:47. The nucleotide sequence of a TYROBP reference mRNA molecule (Isoform 3) is set forth in SEQ ID NO:48. The nucleotide sequence of a TYROBP reference mRNA molecule (Isoform 4) is set forth in SEQ ID NO:49. The nucleotide sequence of a TYROBP reference mRNA molecule (Isoform 5) is set forth in SEQ ID NO:50. The nucleotide sequence of a TYROBP reference mRNA molecule (Isoform 6) is set forth in SEQ ID NO:51. The nucleotide sequence of a TYROBP reference mRNA molecule (Isoform 7) is set forth in SEQ ID NO:52. The nucleotide sequence of a TYROBP reference mRNA molecule (Isoform 8) is set forth in SEQ ID NO:53. The nucleotide sequence of a TYROBP reference mRNA molecule (Isoform 9) is set forth in SEQ ID NO:54 The nucleotide sequence of a TYROBP reference mRNA molecule (Isoform 10) is set forth in SEQ ID NO:55. The nucleotide sequence of a TYROBP reference mRNA molecule (Isoform 11) is set forth in SEQ ID NO:56. The nucleotide sequence of a TYROBP reference mRNA molecule (Isoform 12) is set forth in SEQ ID NO:57. The nucleotide sequence of a TYROBP reference mRNA molecule (Isoform 13) is set forth in SEQ ID NO:58. The nucleotide sequence of a TYROBP reference mRNA molecule (Isoform 14) is set forth in SEQ ID NO:59. The nucleotide sequence of a TYROBP reference mRNA molecule (Isoform 15) is set forth in SEQ ID NO:60. The nucleotide sequence of a TYROBP reference mRNA molecule (Isoform 16) is set forth in SEQ ID NO:61. The nucleotide sequence of a TYROBP reference mRNA molecule (Isoform 17) is set forth in SEQ ID NO:62.

The nucleotide sequence of a TREM2 reference cDNA molecule (Isoform 1) is set forth in SEQ ID NO:21. Referring to SEQ ID NO:21, position 201 is a cytosine, and codon positions 201-203 is a CAG codon.

The nucleotide sequence of a TREM2 reference cDNA molecule (Isoform 2) is set forth in SEQ ID NO:22. Referring to SEQ ID NO:22, position 118 is a cytosine, and codon positions 118-120 is a CAG codon.

The nucleotide sequence of a TREM2 reference cDNA molecule (Isoform 3) is set forth in SEQ ID NO:23. Referring to SEQ ID NO:23, position 131 is a cytosine, and codon positions 131-133 is a CAG codon.

The nucleotide sequence of a TREM2 reference cDNA molecule (Isoform 4) is set forth in SEQ ID NO:24. Referring to SEQ ID NO:24, position 131 is a cytosine, and codon positions 131-133 is a CAG codon.

The nucleotide sequence of a TREM2 reference cDNA molecule (Isoform 5) is set forth in SEQ ID NO:25. Referring to SEQ ID NO:25, position 118 is a cytosine, and codon positions 118-120 is a CAG codon.

The nucleotide sequence of a TREM2 reference cDNA molecule (Isoform 6) is set forth in SEQ ID NO:26. Referring to SEQ ID NO:26, position 201 is a cytosine, and codon positions 131-133 is a CAG codon.

The nucleotide sequence of a TREM2 reference cDNA molecule (Isoform 7) is set forth in SEQ ID NO:27. Referring to SEQ ID NO:27, position 201 is a cytosine, and codon positions 131-133 is a CAG codon.

The nucleotide sequence of a TREM2 reference cDNA molecule (Isoform 8) is set forth in SEQ ID NO:28. Referring to SEQ ID NO:28, position 174 is a cytosine, and codon positions 174-176 is a CAG codon.

The nucleotide sequence of a TREM2 reference cDNA molecule (Isoform 9) is set forth in SEQ ID NO:29. Referring to SEQ ID NO:29, position 191 is a cytosine, and codon positions 191-193 is a CAG codon.

A variant cDNA molecule of TREM2 exists, wherein the cytosine at position 201 according to SEQ ID NO:21 is replaced with thymine, and the CAG codon at positions 201-203 according to SEQ ID NO:21 is replaced with TAG. The nucleotide sequence of this TREM2 variant cDNA molecule is set forth in SEQ ID NO:30.

Another variant cDNA molecule of TREM2 exists, wherein the cytosine at position 118 according to SEQ ID NO:22 is replaced with thymine, and the CAG codon at positions 118-120 according to SEQ ID NO:22 is replaced with TAG. The nucleotide sequence of this TREM2 variant cDNA molecule is set forth in SEQ ID NO:31.

Another variant cDNA molecule of TREM2 exists, wherein the cytosine at position 131 according to SEQ ID NO:23 is replaced with thymine, and the CAG codon at positions 131-133 according to SEQ ID NO:23 is replaced with TAG. The nucleotide sequence of this TREM2 variant cDNA molecule is set forth in SEQ ID NO:32.

Another variant cDNA molecule of TREM2 exists, wherein the cytosine at position 131 according to SEQ ID NO:24 is replaced with thymine, and the CAG codon at positions 131-133 according to SEQ ID NO:24 is replaced with TAG. The nucleotide sequence of this TREM2 variant cDNA molecule is set forth in SEQ ID NO:33.

A variant cDNA molecule of TREM2 exists, wherein the cytosine at position 118 according to SEQ ID NO:25 is replaced with thymine, and the CAG codon at positions 118-120 according to SEQ ID NO:25 is replaced with TAG. The nucleotide sequence of this TREM2 variant cDNA molecule is set forth in SEQ ID NO:34.

Another variant cDNA molecule of TREM2 exists, wherein the cytosine at position 201 according to SEQ ID NO:26 is replaced with thymine, and the CAG codon at positions 131-133 according to SEQ ID NO:26 is replaced with TAG. The nucleotide sequence of this TREM2 variant cDNA molecule is set forth in SEQ ID NO:35.

Another variant cDNA molecule of TREM2 exists, wherein the cytosine at position 201 according to SEQ ID NO:27 is replaced with thymine, and the CAG codon at positions 131-133 according to SEQ ID NO:27 is replaced with TAG. The nucleotide sequence of this TREM2 variant cDNA molecule is set forth in SEQ ID NO:36.

Another variant cDNA molecule of TREM2 exists, wherein the cytosine at position 174 according to SEQ ID NO:28 is replaced with thymine, and the CAG codon at positions 174-176 according to SEQ ID NO:28 is replaced with TAG. The nucleotide sequence of this TREM2 variant cDNA molecule is set forth in SEQ ID NO:37.

Another variant cDNA molecule of TREM2 exists, wherein the cytosine at position 191 according to SEQ ID NO:29 is replaced with thymine, and the CAG codon at positions 191-193 according to SEQ ID NO:29 is replaced with TAG. The nucleotide sequence of this TREM2 variant cDNA molecule is set forth in SEQ ID NO:38.

The nucleotide sequence of a TYROBP reference cDNA molecule (Isoform 1) is set forth in SEQ ID NO:63. The nucleotide sequence of a TYROBP reference cDNA molecule (Isoform 2) is set forth in SEQ ID NO:64. The nucleotide sequence of a TYROBP reference cDNA molecule (Isoform 3) is set forth in SEQ ID NO:65. The nucleotide sequence of a TYROBP reference cDNA molecule (Isoform 4) is set forth in SEQ ID NO:66. The nucleotide sequence of a TYROBP reference cDNA molecule (Isoform 5) is set forth in SEQ ID NO:67. The nucleotide sequence of a TYROBP reference cDNA molecule (Isoform 6) is set forth in SEQ ID NO:68. The nucleotide sequence of a TYROBP reference cDNA molecule (Isoform 7) is set forth in SEQ ID NO:69. The nucleotide sequence of a TYROBP reference cDNA molecule (Isoform 8) is set forth in SEQ ID NO:70. The nucleotide sequence of a TYROBP reference cDNA molecule (Isoform 9) is set forth in SEQ ID NO:71 The nucleotide sequence of a TYROBP reference cDNA molecule (Isoform 10) is set forth in SEQ ID NO:72. The nucleotide sequence of a TYROBP reference cDNA molecule (Isoform 11) is set forth in SEQ ID NO:73. The nucleotide sequence of a TYROBP reference cDNA molecule (Isoform 12) is set forth in SEQ ID NO:74. The nucleotide sequence of a TYROBP reference cDNA molecule (Isoform 13) is set forth in SEQ ID NO:75. The nucleotide sequence of a TYROBP reference cDNA molecule (Isoform 14) is set forth in SEQ ID NO:76. The nucleotide sequence of a TYROBP reference cDNA molecule (Isoform 15) is set forth in SEQ ID NO:77. The nucleotide sequence of a TYROBP reference cDNA molecule (Isoform 16) is set forth in SEQ ID NO:78. The nucleotide sequence of a TYROBP reference cDNA molecule (Isoform 17) is set forth in SEQ ID NO:79.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence. In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a TREM2 nucleic acid molecule comprising a nucleotide sequence that comprises a thymine at a position corresponding to position 1,630 according to SEQ ID NO:2 means that if the nucleotide sequence of the TREM2 genomic nucleic acid molecule is aligned to the sequence of SEQ ID NO:2, the TREM2 sequence has a thymine residue at the position that corresponds to position 1,630 of SEQ ID NO:2. The same applies for TREM2 mRNA molecules comprising a nucleotide sequence that comprises a uracil at a position corresponding to position 201 according to SEQ ID NO:12, and TREM2 cDNA molecules comprising a nucleotide sequence that comprises a thymine at a position corresponding to position 201 according to SEQ ID NO:30. In other words, these phrases refer to a TREM2 nucleic acid molecule that has a nucleotide sequence that comprises a thymine residue that is homologous to the thymine residue at position 1,630 of SEQ ID NO:2 (or wherein the mRNA molecule has a nucleotide sequence that comprises a uracil residue that is homologous to the uracil residue at position 201 of SEQ ID NO:12, or wherein the cDNA molecule has a nucleotide sequence that comprises a thymine residue that is homologous to the thymine residue at position 201 of SEQ ID NO:30).

As described herein, a position within a TREM2 genomic nucleic acid molecule that corresponds to position 1,630 according to SEQ ID NO:2, for example, can be identified by performing a sequence alignment between the nucleotide sequence of a particular TREM2 nucleic acid molecule and the nucleotide sequence of SEQ ID NO:2. A variety of computational algorithms exist that can be used for performing a sequence alignment to identify a nucleotide position that corresponds to, for example, position 1,630 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequences of a TREM2 reference polypeptides are set forth in SEQ ID NO:39 (Isoform 1), SEQ ID NO:42 (Isoform 2).

Referring to SEQ ID NO:39 (Isoform 1), the TREM2 reference polypeptide is 219 amino acids in length. Referring to SEQ ID NO:39, position 33 is glutamine. Referring to SEQ ID NO:39, position 191 is tryptophan.

Referring to SEQ ID NO:42 (Isoform 2), the TREM2 reference polypeptide is 249 amino acids in length. Referring to SEQ ID NO:42, position 63 is glutamine. Referring to SEQ ID NO:42, position 221 is tryptophan.

Referring to SEQ ID NO:40 (Isoform 3), the TREM2 reference polypeptide is 222 amino acids in length.

Referring to SEQ ID NO:41 (Isoform 4), the TREM2 reference polypeptide is 230 amino acids in length.

A set of TREM2 truncated variant polypeptides exists. Referring to SEQ ID NO:43, the TREM2 variant polypeptide is 32 amino acids in length. Referring to SEQ ID NO:43, the TREM2 variant polypeptide is truncated at position 32 and does not contain amino acids at positions corresponding to positions 33-219 of SEQ ID NO:39. Referring to SEQ ID NO:44, the TREM2 variant polypeptide is 62 amino acids in length. Referring to SEQ ID NO:44, the TREM2 variant polypeptide is truncated at position 62 and does not contain amino acids at positions corresponding to positions 63-249 of SEQ ID NO:42.

The amino acid sequences of a TYROBP reference polypeptides are set forth in SEQ ID NO:80 (Isoform 1), SEQ ID NO:81 (Isoform 2), ID NO:82 (Isoform 3), SEQ ID NO:83 (Isoform 4), ID NO:84 (Isoform 5), SEQ ID NO:85 (Isoform 6).

Referring to SEQ ID NO:80 (Isoform 1), the TYROBP reference polypeptide is 101 amino acids in length. Referring to SEQ ID NO:81 (Isoform 2), the TYROBP reference polypeptide is 112 amino acids in length. Referring to SEQ ID NO:82 (Isoform 3), the TYROBP reference polypeptide is 144 amino acids in length. Referring to SEQ ID NO:83 (Isoform 4), the TYROBP reference polypeptide is 102 amino acids in length. Referring to SEQ ID NO:84 (Isoform 5), the TYROBP reference polypeptide is 113 amino acids in length. Referring to SEQ ID NO:85 (Isoform 6), the TYROBP reference polypeptide is 35 amino acids in length.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit Alzheimer's Disease for use in the treatment of Alzheimer's Disease (or for use in the manufacture of a medicament for treating or inhibiting Alzheimer's Disease) in a subject having a TREM2 variant nucleic acid molecule.

In some embodiments, the subject has a TREM2 variant genomic nucleic acid molecule having a nucleotide sequence comprising a thymine at a position corresponding to position 1,630 according to SEQ ID NO:2, or the complement thereof.

In some embodiments, the subject has a TREM2 variant mRNA molecule having a nucleotide sequence comprising: a uracil at a position corresponding to position 201 according to SEQ ID NO:12, or the complement thereof; a uracil at a position corresponding to position 118 according to SEQ ID NO:13, or the complement thereof; a uracil at a position corresponding to position 131 according to SEQ ID NO:14, or the complement thereof; a uracil at a position corresponding to position 131 according to SEQ ID NO:15, or the complement thereof; a uracil at a position corresponding to position 118 according to SEQ ID NO:16, or the complement thereof; a uracil at a position corresponding to position 201 according to SEQ ID NO:17, or the complement thereof; a uracil at a position corresponding to position 201 according to SEQ ID NO:18, or the complement thereof; a uracil at a position corresponding to position 174 according to SEQ ID NO:19, or the complement thereof; or a uracil at a position corresponding to position 191 according to SEQ ID NO:20 or the complement thereof.

In some embodiments, the subject has a TREM2 variant cDNA molecule having a nucleotide sequence comprising: a thymine at a position corresponding to position 201 according to SEQ ID NO:30, or the complement thereof; a thymine at a position corresponding to position 118 according to SEQ ID NO:31, or the complement thereof; a thymine at a position corresponding to position 131 according to SEQ ID NO:32, or the complement thereof; a thymine at a position corresponding to position 131 according to SEQ ID NO:33, or the complement thereof; a thymine at a position corresponding to position 118 according to SEQ ID NO:34, or the complement thereof; a thymine at a position corresponding to position 201 according to SEQ ID NO:35, or the complement thereof; a thymine at a position corresponding to position 201 according to SEQ ID NO:36, or the complement thereof; a thymine at a position corresponding to position 174 according to SEQ ID NO:37, or the complement thereof; or a thymine at a position corresponding to position 191 according to SEQ ID NO:38, or the complement thereof.

The present disclosure also provides therapeutic agents that treat or inhibit Alzheimer's Disease for use in the treatment of Alzheimer's Disease (or for use in the manufacture of a medicament for treating or inhibiting Alzheimer's Disease) in a subject having a TYROBP variant nucleic acid molecule.

In some embodiments, the subject has a TYROBP genomic nucleic acid molecule having a nucleotide sequence comprising any one or more of: 19:35907523:AG:A, 19:35907461:G:A, 19:35907250:G:A, 19:35907729:C:T, 19:35908220:TC:T, 19:35907582:T:C, 19:35908220:T:TC, 19:35907248:CTG:C, 19:35907539:TCC:T, 19:35904569:T:G, 19:35904575:G:C, 19:35904575:G:T, 19:35904622:G:A, 19:35904635:C:T, 19:35907222:TA:T, 19:35907247:G:GCTGTTTCC, 19:35908208:G:T, or 19:35908228:T:C.

In some embodiments, the subject has a TYROBP mRNA molecule having a nucleotide sequence comprising any one or more of: 19:35907523:AG:A, 19:35907461:G:A, 19:35907250:G:A, 19:35907729:C:T, 19:35908220:TC:T, 19:35907582:T:C, 19:35908220:T:TC, 19:35907248:CTG:C, 19:35907539:TCC:T, 19:35904569:T:G, 19:35904575:G:C, 19:35904575:G:T, 19:35904622:G:A, 19:35904635:C:T, 19:35907222:TA:T, 19:35907247:G:GCTGTTTCC, 19:35908208:G:T, or 19:35908228:T:C.

In some embodiments, the subject has a TYROBP cDNA molecule having a nucleotide sequence comprising any one or more of: 19:35907523:AG:A, 19:35907461:G:A, 19:35907250:G:A, 19:35907729:C:T, 19:35908220:TC:T, 19:35907582:T:C, 19:35908220:T:TC, 19:35907248:CTG:C, 19:35907539:TCC:T, 19:35904569:T:G, 19:35904575:G:C, 19:35904575:G:T, 19:35904622:G:A, 19:35904635:C:T, 19:35907222:TA:T, 19:35907247:G:GCTGTTTCC, 19:35908208:G:T, or 19:35908228:T:C.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Rare Loss-of-Function Variants in TREM2 and TYROBP Increase the Risk for Alzheimer's Disease-Related Dementia

In the cohort of 430,998 individuals of European ancestry from the UKB biobank who have exome sequencing data available, 224 TREM2 variants were found that are predicted to alter the encoded protein (e.g., nonsynonymous variants, splice site variants, and frameshifts). To test the role of these variants in Alzheimer's related dementia, two overlapping case cohorts were used, one with 2,446 individuals who have a diagnosis of AD, and a second case cohorts of 59,184 individuals who either have an AD diagnosis or have reported a parent affected with Alzheimer's type dementia (pAD). These two case cohorts were compared to a control group of 371,804 individuals who fulfill neither of these two conditions. Most of the ascertained protein altering TREM2 variants are rare. Among the twelve variants with an alternative allele frequency greater than 1e-4, two variants with significant associations with AD or pAD were found. These comprise the well-established risk variant Arg47His with AD (OR=3.25, P=2e-11) and pAD (OR=1.51, P=2.2e-16), equally established Arg62His variant with AD (OR=1.65, P=4e-5) and pAD (OR=1.17, P=1.3e-7) (Table 1). In addition to these results obtained with an additive risk model, the large sample size allows for testing the effect of these variants on AD risk also under a recessive model. This shows a greatly enhanced risk for homozygotes of either of the two variants (OR=16.5, P=1.3e-4), with a greater risk for individuals who are homozygous for the less frequent Arg47His variant.

TABLE 1
Results for the Arg47His and Arg62His TREM2 variants with AD and pAD in UKB

| CPRA | | | | |
|---|---|---|---|---|
| AAchange | Phenotype | Case Ref \| Het \| Alt | Ctrl Ref \| Het \| Alt | OR_yates |
| 6:41161514:C:T | AD_or_PAD | 58648 \| 529 \| 4 | 369559 \| 2233 \| 2 | 1.511 |
| Arg47His | | | | |
| 6:41161514:C:T | AD | 2402 \| 41 \| 3 | 369559 \| 2233 \| 2 | 3.248 |
| 6:41161469:C:T | AD_or_PAD | 57850 \| 1320 \| 14 | 364613 \| 7155 \| 35 | 1.174 |
| Arg62His | | | | |
| 6:41161469:C:T | AD | 2369 \| 76 \| 1 | 364613 \| 7155 \| 35 | 1.662 |
| | AD_or_PAD | 57322 \| 1844 \| 18 | 362400 \| 9367 \| 37 | 1.251783 |
| | AD | 2326 \| 116 \| 4 | 362400 \| 9367 \| 37 | 1.988137 |

| CPRA | | | | | | | |
|---|---|---|---|---|---|---|---|
| AAchange | Phenotype | OR | CI95 | Pval | OR_rec | CI95_rec | Pval_rec |
| 6:41161514:C:T | AD_or_PAD | 1.51 | 1.37-1.66 | 2.27E−16 | | | |
| Arg47His | | | | | | | |
| 6:41161514:C:T | AD | 3.215 | 2.35-4.3 | 2.00E−11 | 231.4 | 26.4-2840 | 2.67E−06 |
| 6:41161469:C:T | AD_or_PAD | 1.174 | 1.11-1.24 | 1.26E−07 | | | |
| Arg62His | | | | | | | |
| 6:41161469:C:T | AD | 1.651 | 1.3-2.07 | 4.06E−05 | 4.398 | 0.108-26.2 | 0.208 |
| | AD_or_PAD | 1.252 | | 1.74E−17 | | | |
| | AD | 1.992 | 1.68_2.42 | 2.95E−11 | 16.5 | 4.36_46.9 | 0.000152 |

The effect size observed under an additive model are consistent with previous reports for these two established risk variants. In addition, there is a strong risk increase under a recessive model for the two variants.

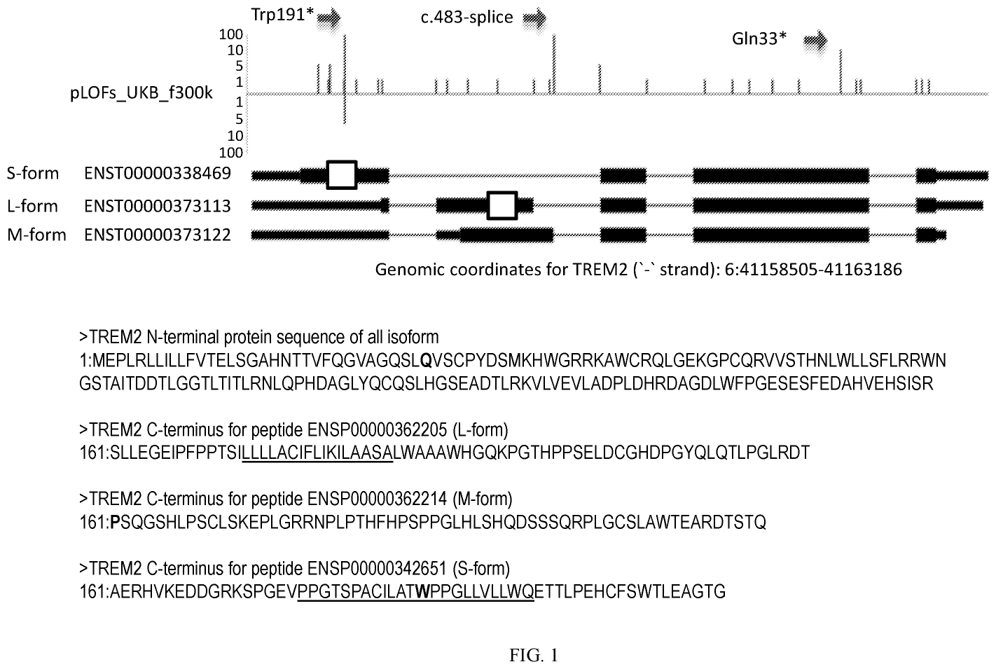

To perform a comprehensive test that includes the less frequent TREM2 variants, a rare variant burden analysis was performed as described earlier (Van Hout et al. 2020). For the rollup of pLoF variants a nominal significant association with pAD was present when including pLoFs with an allele frequency below 1e-4 (OR=2.67, P=0.00014). Surprisingly, no significant effect was seen for the supposedly better powered inclusion of more frequent pLoFs. This result became clearer when inspecting the three most common pLoF variants separately, which showed a risk increasing effect for the Q33X variant, but not the two more frequent Trp191X and the c.483-1G>A splice acceptor variant (Table 2). Importantly, these two latter variants each affect only one of the three different TREM2 splice isoforms, and neither affects the canonical longest transcript (L-form) that encodes the main membrane bound TREM2 isoform (FIG. 1). Thus, one might hypothesize that loss of the canonical TREM2 transcript leads to AD, but not of the two other isoforms, which have an entirely different C-terminal sequence. Consistent with this hypothesis, a similar association with pAD was observed in an aggregate analysis of pLoF variants that are predicted to abolish the L-form transcript (OR=3.7, P=1.6e-5) (Table 3). Notably, the above TREM2 Q33X variant has been previously identified as cause of autosomal recessive PLOSL (Soragna et al. 2003), which would be consistent with an increased AD risk for individuals who are heterozygous for PLOSL mutations.

TABLE 2
Results for the three most frequent TREM2 pLoF variants

| CPRA | | Case | Ctrl |
|---|---|---|---|
| AAchange | Phenotype | Ref\|Het\|Alt | Ref\|Het\|Alt |
| 6:41161557:G:A | AD_or_PAD | 59171\|13\|0 | 371790\|14\|0 |
| Gln33* | | | |
| 6:41161557:G:A | AD | 2445\|1\|0 | 371790\|14\|0 |

| CPRA | | | | | |
|---|---|---|---|---|---|
| AAchange | Phenotype | OR_yates | OR | CI95 | Pval |
| 6:41161557:G:A | AD_or_PAD | 5.849 | 5.834 | 2.52_13.4 | 1.85E−05 |
| Gln33* | | | | | |
| 6:41161557:G:A | AD | 15.73 | 10.86 | 0.257_71.3 | 0.09368 |

Only the less frequent Q33X variant shows any evidence for association. This variant affects all three TREM2 transcripts, in contrast to the other two variants which only affect one transcript each.

TABLE 3
Burden test of TREM2 pLoF variants that affect the L-form

| | Case | Ctrl | |
|---|---|---|---|
| Phenotype | Ref\|Het\|Alt | Ref\|Het\|Alt | OR_yates |
| AD_or_PAD | 59164\|20\|0 | 371770\|34\|0 | 3.733 |
| AD | 2445\|1\|0 | 371770\|34\|0 | 6.609 |

| Phenotype | OR | CI95 | Pval |
|---|---|---|---|
| AD_or_PAD | 3.696 | 2.02_6.61 | 1.61E−05 |
| AD | 4.472 | 0.11_26.7 | 0.2051 |

Association of variants in TYROPB, a gene acting downstream of TREM2 was tested next. Two variants attained an allele frequency greater than 1e-4 and both showed associations with AD and/or pAD, consistent with an association of TYROBP with AD risk. Additional support for an effect of TYROBP loss-of-function comes from the burden test results, which show an increased risk of AD in the aggregate analysis of protein altering variants under different filtering schemes. For the aggregate analysis TYROBP pLoF variants there is evidence for an association with AD (OR=7.5, P=7e-4) as wells as pAD (OR=2.1 P=2e-4) (Table 4).

TABLE 4
Burden test of TYROBP pLoF variants

| | Case | Ctrl | |
|---|---|---|---|
| Phenotype | Ref\|Het\|Alt | Ref\|Het\|Alt | OR_yates |
| AD_or_PAD | 59149\|35\|0 | 371702\|102\|0 | 2.176 |
| AD | 2441\|5\|0 | 371702\|102\|0 | 8.163 |

| Phenotype | OR | CI95 | Pval |
|---|---|---|---|
| AD_or_PAD | 2.156 | 1.42_3.19 | 0.0002501 |
| AD | 7.458 | 2.37_18 | 0.0007285 |

Finally, adjoined burden test across pLoF variants in TYROBP and TREM2 was performed, but including for TREM2 only those pLoFs that affect the transcript L-form. All carriers of these variants are heterozygotes. This joined analysis of pLoF variants in either of the two genes shows enhanced statistical significance (OR=2.5, P=6.7e-8), further confirming that loss TREM2/TYROBP signaling increases AD risk (Table 5).

TABLE 5
Joint burden test of TREM2 and TYROBP pLoF variants

| | Case | Ctrl | | | | |
|---|---|---|---|---|---|---|
| Phenotype | Ref \| Het \| Alt | Ref \| Het \| Alt | OR_yates | OR | CI95 | Pval |
| AD_or_PAD | 59129 \| 55 \| 0 | 371668 \| 136 \| 0 | 2.555 | 2.541 | 1.82_3.5 | 6.75E−08 |
| AD | 2440 \| 6 \| 0 | 371668 \| 136 \| 0 | 7.245 | 6.713 | 2.42_15 | 0.000373 |

Using UKB exome sequencing data, TREM2/TYROBP pLoF variants were found to increase the risk of Alzheimer's related dementia. This observation supports the notion that reduced signaling through the TREM2/TYROBP is an important cause of AD related dementia. These results further show that the alternative TREM2 transcript isoforms differ in their functional relevance, and that AD risk is specifically increased by the abolishment of the long isoform.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having Alzheimer's Disease or at risk of developing Alzheimer's Disease, the method comprising administering a Triggering Receptor Expressed On Myeloid Cells 2 (TREM2) agonist to the subject.

2. The method according to claim 1, wherein the subject has a TREM2 loss-of-function variant nucleic acid molecule.

3. The method according to claim 2, wherein the TREM2 loss-of-function variant nucleic acid molecule is a missense variant, splice-site variant, a stop-gain variant, a start-loss variant, a stop-loss variant, a frameshift variant, or an in-frame indel variant, or a variant that encodes a truncated TREM2 predicted loss-of-function polypeptide.

4. A method of treating a subject with a therapeutic agent that treats or inhibits Alzheimer's Disease, wherein the subject has Alzheimer's Disease or is at risk of developing Alzheimer's Disease, the method comprising:
determining whether the subject has a Triggering Receptor Expressed On Myeloid Cells 2 (TREM2) variant nucleic acid molecule by:
obtaining or having obtained a biological sample from the subject; and
performing or having performed a sequence analysis on the biological sample to determine if the subject has a genotype comprising the TREM2 variant nucleic acid molecule; and
when the subject is TREM2 reference, then administering or continuing to administer to the subject the therapeutic agent that treats or inhibits Alzheimer's Disease in a standard dosage amount and/or a TREM2 agonist; and
when the subject is heterozygous or homozygous for the TREM2 variant nucleic acid molecule, then administering or continuing to administer to the subject the therapeutic agent that treats or inhibits Alzheimer's Disease in an amount that is the same as or greater than a standard dosage amount and/or a TREM2 agonist;
wherein the presence of a genotype having the TREM2 variant nucleic acid molecule indicates the subject has an increased risk of developing Alzheimer's Disease or developing a more severe form of Alzheimer's Disease.

5. The method according to claim 4, wherein the subject is TREM2 reference, and the subject is administered or continued to be administered the therapeutic agent that treats or inhibits Alzheimer's Disease in a standard dosage amount and/or a TREM2 agonist.

6. The method according to claim 4, wherein the subject is heterozygous or homozygous for the TREM2 variant nucleic acid molecule, and the subject is administered or continued to be administered the therapeutic agent that treats or inhibits Alzheimer's Disease in an amount that is the same as or greater than a standard dosage amount and/or a TREM2 agonist.

7. The method according to claim 4, wherein the TREM2 variant nucleic acid molecules comprise any one or more of: 6:41159107:C:T, 6:41158691:C:T, 6:41161557:G:A, 6:41158639:GT:G, 6:41158662:G:A, 6:41159790:A:G, 6:41161285:CT:C, 6:41159068:T:G, 6:41163053:GA:G, 6:41158690:C:T, 6:41161340:GC:G, 6:41158659:C:A, 6:41158715:C:CT, 6:41158894:AC:A, 6:41158994:G:A, or 6:41161587:CTG:C.

8. The method according to claim 4, wherein the TREM2 variant nucleic acid molecule is a nucleic acid molecule encoding Gln33STOP.

9. The method according to claim 7, wherein the TREM2 variant nucleic acid molecule is:
a genomic nucleic acid molecule having a nucleotide sequence comprising a thymine at a position corresponding to position 1,630 according to SEQ ID NO:2;
an mRNA molecule having a nucleotide sequence comprising: a uracil at a position corresponding to position 201 according to SEQ ID NO:12, a uracil at a position corresponding to position 118 according to SEQ ID NO:13, a uracil at a position corresponding to position 131 according to SEQ ID NO:14, a uracil at a position corresponding to position 131 according to SEQ ID NO:15, a uracil at a position corresponding to position 118 according to SEQ ID NO:16, a uracil at a position corresponding to position 201 according to SEQ ID NO:17, a uracil at a position corresponding to position 201 according to SEQ ID NO:18, a uracil at a position corresponding to position 174 according to SEQ ID NO:19, or a uracil at a position corresponding to position 191 according to SEQ ID NO:20; or
a cDNA molecule produced from an mRNA molecule, wherein the cDNA molecule has a nucleotide sequence comprising: a thymine at a position corresponding to position 201 according to SEQ ID NO:30, a thymine at a position corresponding to position 118 according to SEQ ID NO:31, a thymine at a position corresponding to position 131 according to SEQ ID NO:32, a thymine at a position corresponding to position 131 according to SEQ ID NO:33, a thymine at a position corresponding to position 118 according to SEQ ID NO:34, a thymine at a position corresponding to position 201 according to SEQ ID NO:35, a thymine at a position corresponding to position 201 according to SEQ ID NO:36, a thymine at a position corresponding to position 174 according to SEQ ID NO:37, or a thymine at a position corresponding to position 191 according to SEQ ID NO:38.

10-47. (correspond)

48. A method of treating a subject with a therapeutic agent that treats or inhibits Alzheimer's Disease, wherein the subject has Alzheimer's Disease or is at risk of developing Alzheimer's Disease, the method comprising:
determining whether the subject has a Transmembrane Immune Signaling Adaptor (TYROBP) variant nucleic acid molecule by:
obtaining or having obtained a biological sample from the subject; and
performing or having performed a sequence analysis on the biological sample to determine if the subject has a genotype comprising the TYROBP variant nucleic acid molecule; and
when the subject is TYROBP reference, then administering or continuing to administer to the subject the therapeutic agent that treats or inhibits Alzheimer's Disease in a standard dosage amount; and
when the subject is heterozygous or homozygous for the TYROBP variant nucleic acid molecule, then administering or continuing to administer to the subject the therapeutic agent that treats or inhibits Alzheimer's Disease in an amount that is the same as or greater than a standard dosage amount;
wherein the presence of a genotype having the TYROBP variant nucleic acid molecule indicates the subject has an increased risk of developing Alzheimer's Disease or developing a more severe form of Alzheimer's Disease.

49. The method according to claim 48, wherein the subject is TYROBP reference, and the subject is administered or continued to be administered the therapeutic agent that treats or inhibits Alzheimer's Disease in a standard dosage amount.

50. The method according to claim 48, wherein the subject is heterozygous or homozygous for the TYROBP variant nucleic acid molecule, and the subject is administered or continued to be administered the therapeutic agent that treats or inhibits Alzheimer's Disease in an amount that is the same as or greater than a standard dosage amount.

51. The method according to claim 48, wherein the TYROBP variant nucleic acid molecule is a missense variant, a splice-site variant, a stop-gain variant, a start-loss variant, a stop-loss variant, a frameshift variant, or an in-frame indel variant, or a variant that encodes a truncated TYROBP predicted loss-of-function polypeptide.

52. The method according to claims 48, wherein the TYROBP variant nucleic acid molecules comprise any one or more of: 19:35907523:AG:A, 19:35907461:G:A, 19:35907250:G:A, 19:35907729:C:T, 19:35908220:TC:T, 19:35907582:T:C, 19:35908220:T:TC, 19:35907248:CTG:C, 19:35907539:TCC:T, 19:35904569:T:G, 19:35904575:G:C, 19:35904575:G:T, 19:35904622:G:A, 19:35904635:C:T, 19:35907222:TA:T, 19:35907247:G:GCTGTTTCC, 19:35908208:G:T, or 19:35908228:T:C.

53-77. (canceled)
